(19) United States
(12) Patent Application Publication
(10) Pub. No.: US 2009/0203817 A1
(43) Pub. Date: Aug. 13, 2009

(54) STABILIZED SPANDEX COMPOSITIONS

(76) Inventors: Joseph A. Stretanski, Stamford, CT (US); Thomas Sassi, Stamford, CT (US); Ram B. Gupta, Stamford, CT (US)

Correspondence Address: Cytec Industries Inc., 1937 West Main Street, P.O. Box 60, Stamford, CT, 06904-0060, US

(21) Appl. No.: 12/297,032

(22) PCT Filed: Apr. 12, 2007
(86) PCT No.: PCT/US2007/009003

§ 371 Date: Oct. 13, 2008

Related U.S. Application Data
(60) Provisional application No. 60/744,766, filed on Apr. 13, 2006.
(60) Provisional application No. 60/807,668, filed on Jul. 18, 2006.

(51) Int. Cl. C08K 5/3492; C08K 5/36; C07C 321/16; C07C 321/26; C07D 251/32

(52) U.S. Cl. 524/100; 524/289; 560/15; 560/17; 544/192

(57) ABSTRACT

Compounds of the formula (I) as defined herein are useful for stabilizing spandex. Stabilized spandex compositions are obtained by intermixing spandex polymer or prepolymer with stabilizing amounts of one or more compounds of the formula (I). Preferred stabilized spandex polymer or prepolymer compositions exhibit improved color stability.

### BACKGROUND OF THE INVENTION

1. Field of the Invention

This invention relates to stabilized spandex polymer or prepolymer compositions and methods for making them.

2. Description of the Related Art

Spandex is an elastic polymeric material that is widely used in the manufacture of a variety of products, such as sport and exercise apparel. In many of these applications, it is desirable to stabilize various properties of the spandex polymer or prepolymer because it is known that unstabilized spandex polymer or prepolymer undergoes degradation under certain conditions. For example, unstabilized spandex polymer or prepolymer discolors when subjected to prolonged high temperatures during high temperature processing to form spandex fibers and/or exposure to high levels of atmospheric smog containing oxides of nitrogen (NOₓ).

A number of references disclose compounds that are said to be useful for stabilizing spandex polymer or prepolymer. For example, certain compounds of the type shown in TABLE 1 have been disclosed in the indicated references as being spandex stabilizers.

TABLE 1

| Reference | Compound type |
|---|---|
| JP 11-323662 | Hindered phenols and thioesters |
| U.S. Pat. No. 2,999,839 | Aliphatic amines and titanium dioxide |
| U.S. Pat. No. 3,386,942 | Hindered phenols and thioesters |
| U.S. Pat. No. 3,553,290 | Condensates of substituted phenols and divinyl benzenes |
| U.S. Pat. No. 3,573,251 | Substituted phenols and organic phosphites |
| U.S. Pat. No. 3,428,711 | Hindered polymeric tertiary amines |
| U.S. Pat. No. , 4,340,527 | Zinc oxide |
| U.S. Pat. No. 4,548,975 | Hindered phenols and dialkyl phenyl phosphites |
| U.S. Pat. No. 5,626,960 | Mixtures of huntite and hydromagnesite |
| U.S. Pat. No. 5,028,642 | Zinc oxide and sugars |

Although certain compounds of the type shown in TABLE 1 are effective as spandex polymer or prepolymer stabilizers, other compounds of the same type are less effective or ineffective. In this respect spandex stabilization is similar to the general art of polymer stabilization, which is recognized as being relatively unpredictable. For example, in the introduction to the chapter entitled “Stabilization,” the Encyclopedia of Polymer Science and Technology (3^{rd} Ed. 2003) states: “Apart from cost and customer specifications, the amount of protection offered by antioxidants and stabilizers can vary enormously depending on the chemical structure of polymers, their physical and morphological characteristics, the manufacturing process, and service conditions of end-use articles.”

There are many references in the art to compounds that are said to be useful as stabilizers for a variety of polymers. Examples of such references and some of the disclosed compound types are shown in TABLE 2.

TABLE 2

| Reference | Compound type |
|---|---|
| U.S. Pat. No. 2,915,496 | Hindered phenols and organic phosphites |
| U.S. Pat. No. 3,085,991 | Substituted phenols |
| U.S. Pat. No. 3,238,178 | Trialkylated phenols and thioorganic esters |
| U.S. Pat. No. 3,285,855 | Esters containing an alkylhydroxyphenyl group |
| U.S. Pat. No. 3,441,575 | Esters of di(lower)alkylhydroxyphenyl alkanoic acids |
| U.S. Pat. No. 3,536,661 | Sulfur-containing hindered phenols |
| U.S. Pat. No. 3,660,352 | Sulfur-containing hindered phenols |
| U.S. Pat. No. 3,795,700 | Esters of 4-alkyl-2,6-dimethyl-3-hydroxybenzyl alcohol |
| U.S. Pat. No. 3,923,869 | Alkyl-substituted hydroxybenzyl esters of 3,3′-dipropionic acid |
| U.S. Pat. No. 4,857,572 | Sulfur-containing hindered phenols |
| U.S. Pat. No. 5,008,459 | Sulfur-containing hindered phenols |
| U.S. Pat. No. 5,116,894 | Substituted triazines and alkylthiomethylphenols |
| U.S. Pat. No. 5,276,258 | Mercaptomethylphenols |
| U.S. Pat. No. 5,376,290 | Sulfur-containing hindered phenols |
| U.S. 2003/0191217 | Polyol connecting at multiple phenolic groups |

Sulfur-containing hindered phenols are disclosed as stabilizers for various polymers in a number of references, including several of the references mentioned above and in the following: EP 0 035 473 and EP 1 146 038; and U.S. Pat. Nos. 3,584,083; 3,694,440; 3,704,326; 3,763,094; 3,810,869; 3,810,929; 3,984,460; 3,988,363; 3,998,863; 4,132,702; 4,163,008; 4,171,298; 4,187,246; 4,305,868; 4,333,868; 4,439,615; 4,521,559; 4,694,102; 4,772,651; 4,889,883; and 4,954,275.

As noted above, there is enormous variation in the amount of protection offered by stabilizers, depending on the nature of the polymer to be stabilized. Because of this enormous variation, those skilled in the art understand that stabilizers for other polymers are not necessarily stabilizers for spandex. The art sometimes refers in a general way to certain compounds or types of compounds as being polymer stabilizers, and/or refers to the ability of such compounds or types to stabilize particular polymers. However, those skilled in the art would not have expected that such compounds or types would stabilize spandex. For example, art published many years ago may seem to suggest that compounds and types of compounds found to be effective for stabilizing a particular polymer other than spandex would also be effective for stabilizing every other type of polymer, including without limitation, spandex. Other more recent references provide particular stabilization data for particular polymers and, without providing data or scientific reasoning, speculate that those stabilizers might also be effective for stabilizing many other types of polymers. However, such unsupported conjecture, especially when taken in the context of the huge field of polymer stabilization art provides little or no guidance to those skilled in the art seeking to identify effective stabilizers for spandex.

There continues to be a need for effective stabilizers for spandex, methods for incorporating those stabilizers into spandex, and stabilized spandex polymer or prepolymer compositions.

### SUMMARY OF THE INVENTION

Embodiments of the present invention are directed to stabilized spandex polymer or prepolymer compositions comprising stabilizing amounts of the compositions described herein.

For example, an embodiment provides a stabilized spandex polymer or prepolymer composition comprising spandex polymer or prepolymer and a stabilizing amount of a compound of formula (I):

LEₙ

where E is an end group, L is a linking group, and n is an integer in the range of 1 to 10 that represents the number of E end groups attached to the linking group L. Each of the E end groups are independently represented by the formula (E-1)

-{A(CH₂)ₘ₂—S}ₐ(CH₂)ₘ₁—[CZQ]_{q}—Ar where q=0-10 (E-1)

where a is zero or one. In an embodiment, for a=zero in formula (E-1), n is 2 and the linking group L is represented by the formula (L-1) or (L-2):

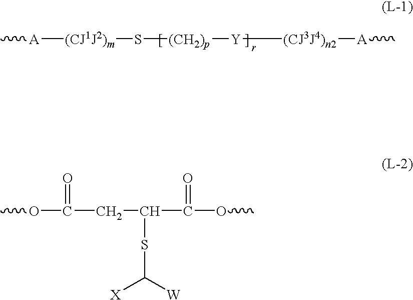

In another embodiment, for a=zero in formula (E-1), n is 2 and the lining group L is represented by the formula (L-1a):

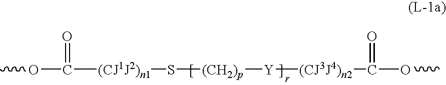

For a=one in formula (E-1), n=2 to 10 and the linking group L is an n-valent optionally substituted C₁-C₃₀ hydrocarbyl group.

In another embodiment when n=1, the linking group

L=H—(CJ¹J²)ₙ₁-S—[(CH₂)ₚY]ᵣ—(CJ³J⁴)ₙ₂-A-

In the formulae (I), (E-1), (L-1) and (L-2), A represents an ester linkage; p is an integer in the range of 2 to 4; r is zero or an integer in the range of 1 to 3; Y is O or S; X and W are each independently H, Ar¹(CH₂)ₙ₃—, or Ar²(CH₂)ₙ₄—O₂C—(CH₂)ₙ₅—; J¹, J², J³, and J⁴ are each independently H, C₁₋₁₀ alkyl or C₆₋₁₀ aryl; and n1, n2, and m2 are each independently an integer in the range of 1 to 20. In the formulae (I), (E-1), (L-1) and (L-2), n1, n2, and m2 are selected so that the compound of formula (I) does not include a covalent bond between sulfur and oxygen; n3, n4, n5, and m1 are each independently zero or an integer in the range of 1 to 20; each Z is independently an optionally substituted aryl or H; each Q is independently an optionally substituted C₁₋₆ alkyl or H; and Ar, Ar¹, and Ar² are each independently selected from the formulae (Ar-1), (Ar-2), (Ar-3) and, optionally, (Ar-4):

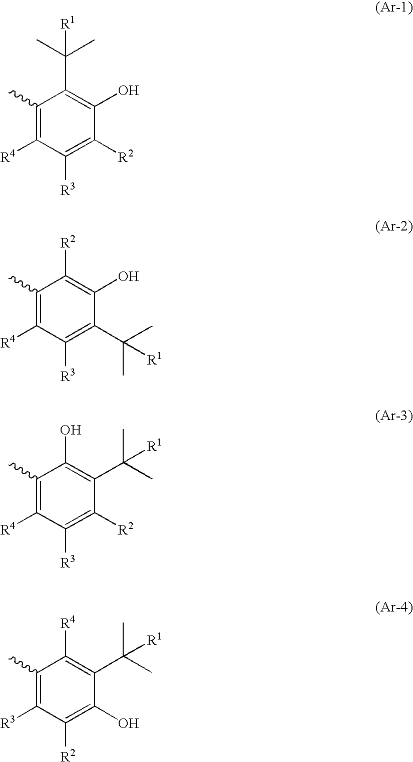

In the formulae (Ar-1), (Ar-2), (Ar-3) and (Ar-4), R¹ is selected from C₁-C₆ alkyl, C₆-C₁₀ aryl and C₇-C₁₂ arylalkyl; and R², R³ and R⁴ are each independently H or C₁-C₆ alkyl. Preferably, R² is H or C₁-C₆ n-alkyl. In a preferred embodiment, Z=H, Q=H, a=o and m1=0.

In some embodiments, formula (I) does not include the following compounds:

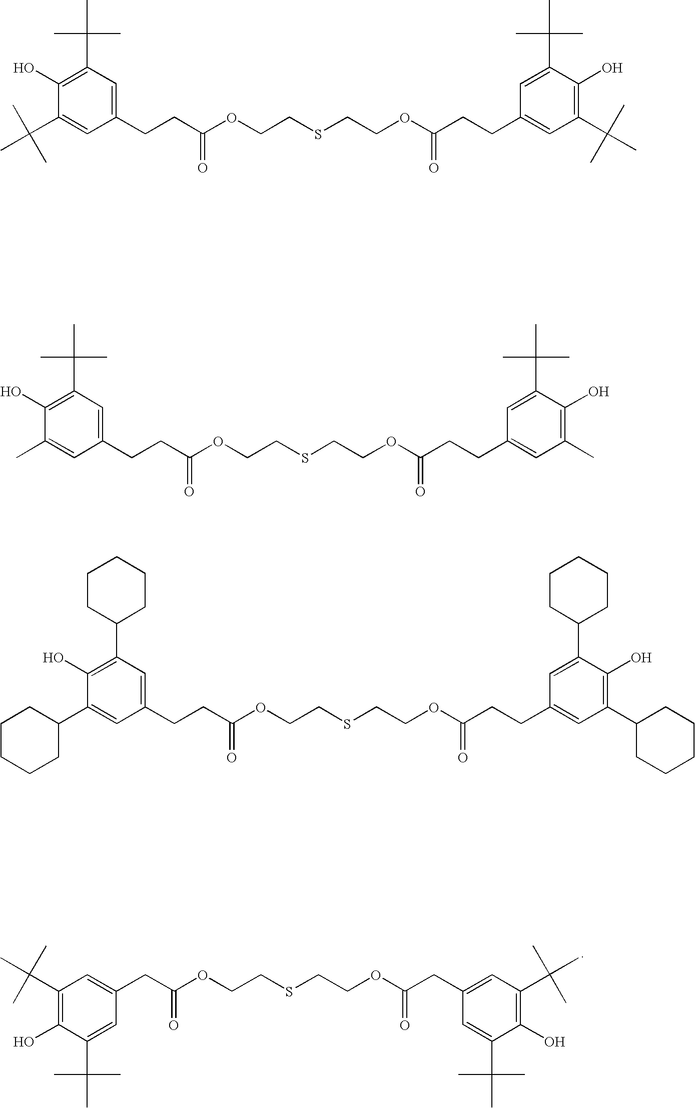

Another embodiment provides a method for making a stabilized spandex polymer or prepolymer composition. The method comprises intermixing spandex polymer or prepolymer and a stabilizing amount of the compound of formula (I).

Other embodiments of the present invention are directed to certain sulfur-containing hindered phenol antioxidants of the formula (I). For example, an embodiment provides a compound of the formula (AO-2):

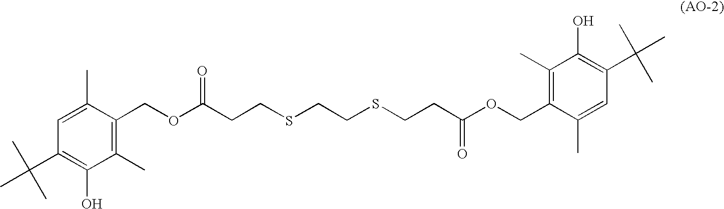

Another embodiment provides a composition comprising a polymer and the compound of the formula (AO-2). In a preferred embodiment, the polymer is spandex polymer or prepolymer.

Another embodiment provides a compound of the formula (AO-3):

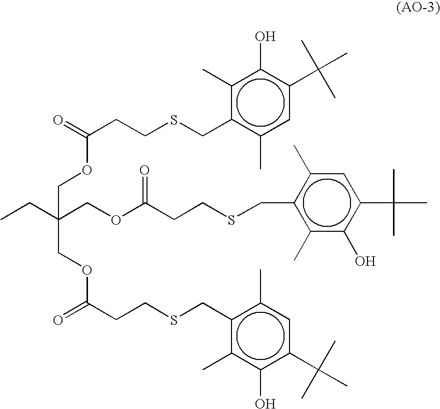

Another embodiment provides a composition comprising a polymer and a compound of the formula (AO-3). In a preferred embodiment, the polymer is spandex polymer or prepolymer.

Another embodiment provides a compound of the formula (AO-9):

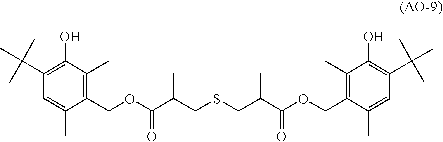

Another embodiment provides a composition comprising a polymer and a compound of the formula (AO-9). In a preferred embodiment, the polymer is spandex polymer or prepolymer.

Another embodiment provides a composition of the formula (AO-10):

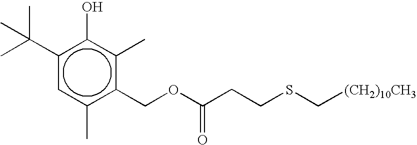

Another embodiment provides a composition of the formula (AO-11):

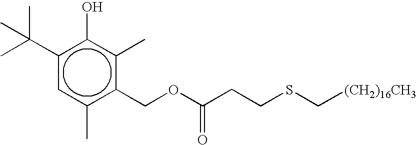

Another embodiment provides a composition of the formula (AO-12):

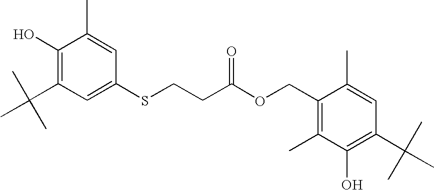

Another embodiment provides a composition of the formula (AO-13):

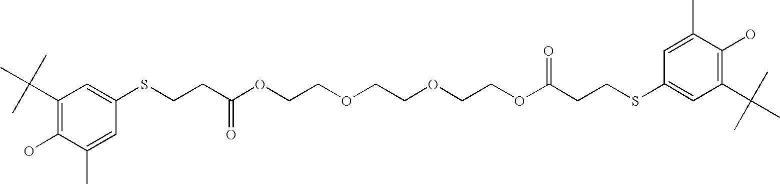

These and other embodiments are described in greater detail below.

### DETAILED DESCRIPTION OF THE PREFERRED EMBODIMENTS

It has now been discovered that compounds of the formula (I) are stabilizers for spandex polymer or prepolymer. For example, when incorporated in stabilizing amounts, preferred compounds of the formula (I) protect spandex polymer or prepolymer against thermo-oxidative yellowing and/or NOₓ discoloration to a degree that is significantly greater than the protection provided by traditional antioxidants. Embodiments of the present invention provide stabilized spandex polymer or prepolymer compositions comprising compounds of the formula (I), optionally further comprising co-additives that may include, but are not limited to, traditional antioxidants, NOₓ stabilizers, UV absorbers, and/or anti-chlor agents, as well as methods of making such compositions. Additional embodiments provide certain novel compounds of the formula (I) and polymer compositions comprising those compounds. The examples provided below illustrate performance improvements that may be obtained in spandex polymer or prepolymer compositions comprising stabilizing amounts of compounds of the formula (I).

An embodiment provides a stabilized spandex polymer or prepolymer composition comprising spandex polymer or prepolymer and stabilizing amount of a compound of formula (I). For example, in an embodiment of such a stabilized spandex polymer or prepolymer composition, the compound of formula (I) is a compound of the formula (Ia):

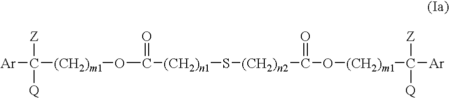

It will be understood by those skilled in the art that the compound of the formula (Ia) is a compound of the formula (I) in which a in formula (E-1) is zero, n in formula (I) is 2, the linking group L in formula (I) is represented by formula (L-1) in which J¹, J², J³, and J⁴ are H, and r and A in formula (L-1) are selected so that L-1 is represented by the formula (L-1b):

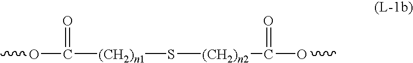

Compound (AO-1), 3,3′-thiobispropanoic acid, bis[[4-(1,1-dimethylethyl)-3-hydroxy-2,6-dimethylphenyl]methyl]ester, is an example of a compound of the formula (Ia):

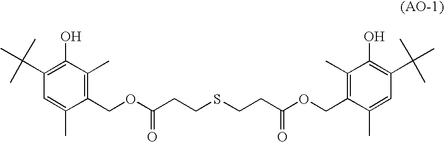

In another embodiment of the stabilized spandex polymer or prepolymer composition, the compound of formula (I) is a compound of the formula (Ib):

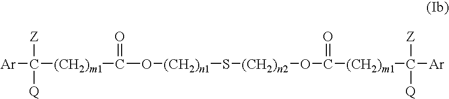

It will be understood by those skilled in the art that the compound of the formula (Ib) is a compound of the formula (I) in which a in formula (E-1) is zero, n in formula (I) is 2, the linking group L in formula (I) is represented by formula (L-1) in which J¹, J², J³, and J⁴ are H, and r and A in formula (L-1) are selected so that L-1 is represented by the formula (L-1c):

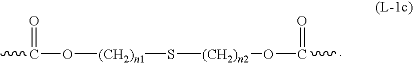

In another embodiment of the stabilized polymer composition, the compound of formula (I) is a compound of the formula (Ic):

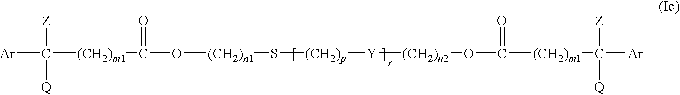

It will be understood by those skilled in the art that the compound of the formula (Ic) is a compound of the formula (I) in which a in formula (E-1) is zero, n in formula (I) is 2, the linking group L in formula (I) is represented by formula (L-1) in which J¹, J², J³, and J⁴ are H, r in formula (L-1) is 1 to 3, and A in formula (L-1) is selected so that L-1 is represented by the formula (L-1d):

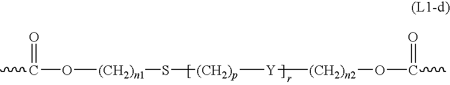

In another embodiment of the stabilized spandex polymer or prepolymer composition, the compound of formula (I) is a compound of the formula (Id):

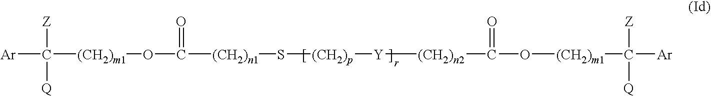

It will be understood by those skilled in the art that the compound of the formula (Id) is a compound of the formula (I) in which a in formula (E-1) is zero, n in formula (I) is 2, the linking group L in formula (I) is represented by formula (L-1) in which J¹, J², J³, and J⁴ are H, r in formula (L-1) is 1 to 3, and A in formula (L-1) is selected so that L-1 is represented by the formula (L-1e):

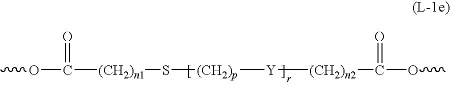

Compound (AO-2) is an example of a compound of the formula (Id):

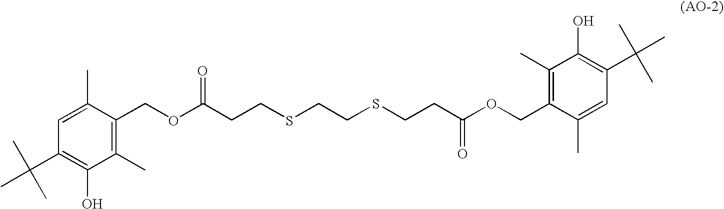

In another embodiment of the stabilized spandex polymer or prepolymer composition, the compound of formula (I) is a compound of the formula (Ie):

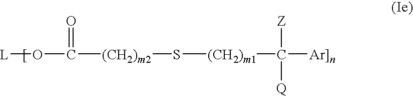

It will be understood by those skilled in the art that the compound of the formula (Ie) is a compound of the formula (I) in which a in formula (E-1) is one, n in formula (I) is an integer in the range of 2 to 10, the linking group L in formula (I) an n-valent optionally substituted C₁-C₃₀ hydrocarbyl group, and A in formula (E-1) is selected so that (E-1) is represented by the formula (E-1e):

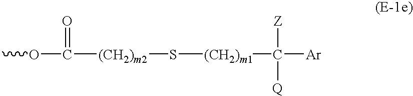

The linking group L may be optionally substituted with one or more groups, each independently selected from alkyl, cycloalkyl, aryl, fused aryl, heterocyclyl, heteroaryl, hydroxy, alkoxy, aryloxy, mercapto, alkylthio, arylthio, cyano, halo, carbonyl, thiocarbonyl, alkoxycarbonyl, nitro, silyl, trihalomethanesulfonyl, trifluoromethyl, and amino. Compounds (AO-3) and (AO-8) are examples of compounds of the formula (Ie):

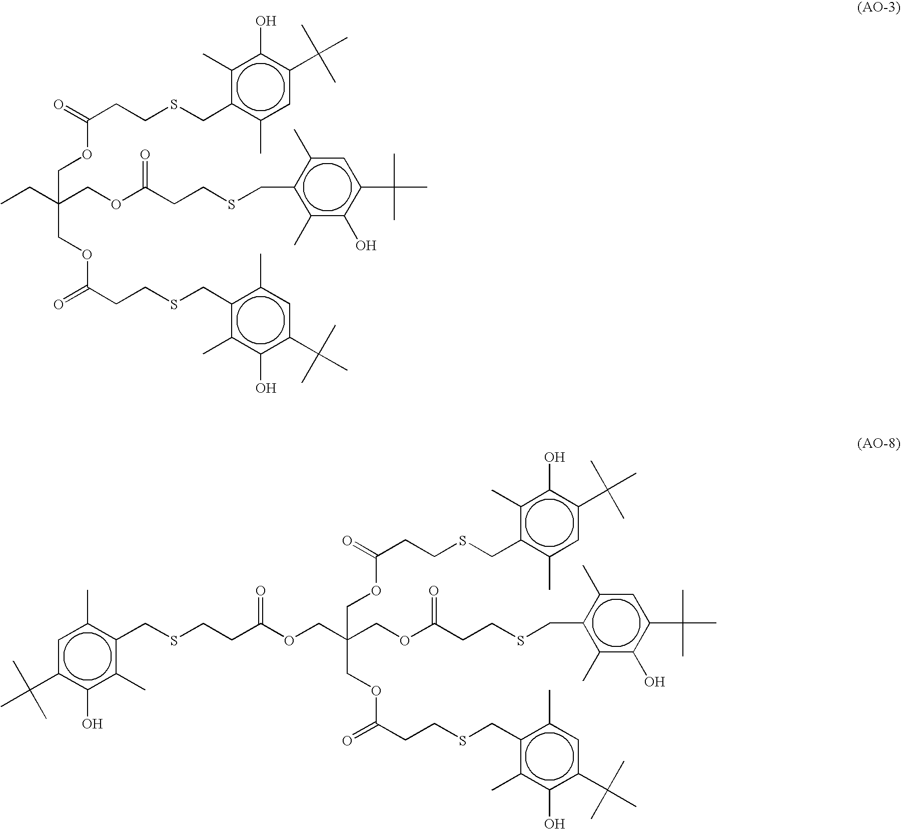

In another embodiment of the stabilized spandex polymer or prepolymer composition, the compound of formula (I) is a compound of the formula (If):

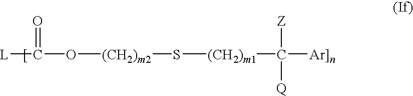

It will be understood by those skilled in the art that the compound of the formula (If) is a compound of the formula (I) in which a in formula (E-1) is one, n in formula (I) is an integer in the range of 2 to 10, the linking group L in formula (I) is an n-valent optionally substituted C₁-C₃₀ hydrocarbyl group, and A in formula (E-1) is selected so that (E-1) is represented by the formula (E-1f):

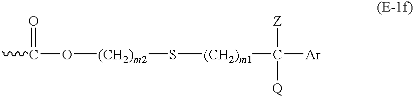

In another embodiment of the stabilized spandex polymer or prepolymer composition, the compound of formula (I) is a compound of the formula (Ig):

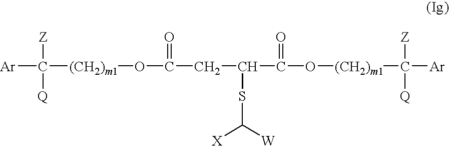

It will be understood by those skilled in the art that the compound of the formula (Ig) is a compound of the formula (I) in which in which a in formula (E-1) is zero, the linking group L in formula (I) is represented by formula (L-2), and n in formula (I) is 2. In a preferred embodiment of formula (Ig), Z=Q=H and m1=0.

In formula (I), J¹, J², J³, and J⁴ are each independently H, C₁₋₁₀ alkyl or C₆₋₁₀ aryl. In a preferred embodiment, J¹, J², J³, and J⁴ are each independently H or methyl. Compound (AO-9) is an example of a compound of the formula (I) in which n1 and n2 are two, one J¹ is H and the other is methyl, and one J³ is H and the other is methyl:

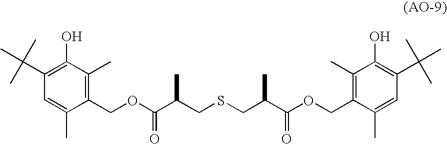

In another embodiment of the stabilized spandex polymer or prepolymer composition, the compound of formula (I) is a compound of

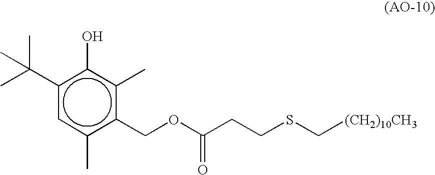

In another embodiment of the stabilized spandex polymer or prepolymer composition, the compound of formula (I) is a compound of

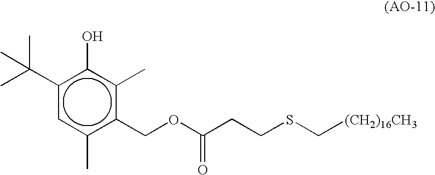

In another embodiment of the stabilized spandex polymer or prepolymer composition, the compound of formula (I) is a compound of

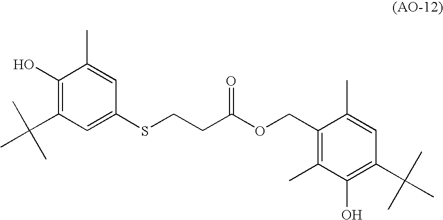

In another embodiment of the stabilized spandex polymer or prepolymer composition, the compound of formula (I) is a compound of

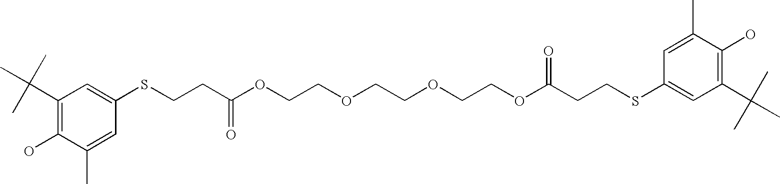

In another embodiment of the stabilized spandex polymer or prepolymer the antioxidant comprises an antioxidant complex comprised of

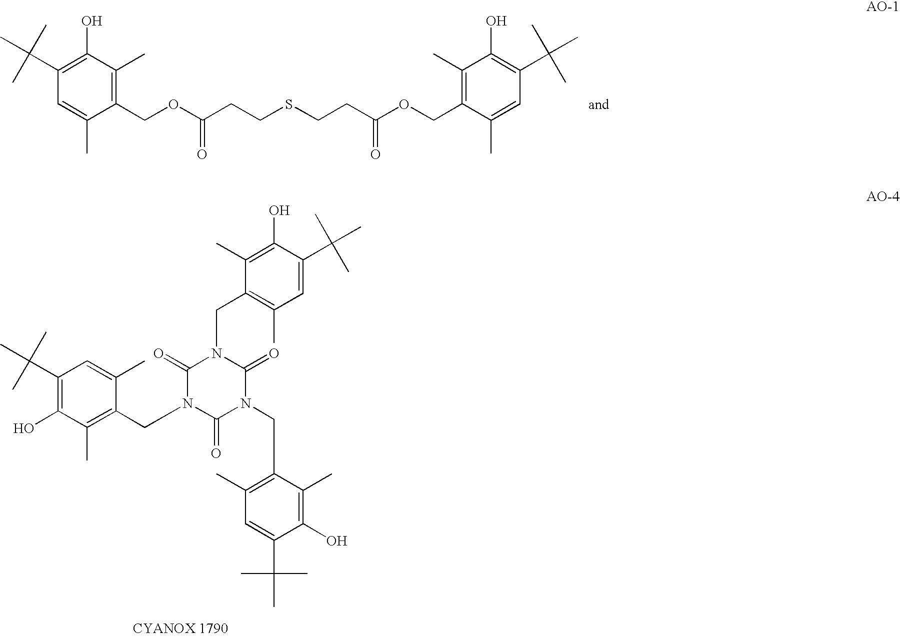

The antioxidant complex is prepared by co-crystallization from a solution containing AO-1 and AO-4 and the ratio of AO-1 to AO-4 is from about 4:1 to about 1:4, preferably about 1:1 and the melting point of the complex is in the range of from about 155° C. and 165° C.

Other compounds encompassed by formula (I) include, but are not limited to, Acetic acid, 2,2′-thiobis-, bis[[4-(1,1-dimethylethyl)-3-hydroxy-2,6-dimethylphenyl]methyl]ester; Propanoic acid, 3-[[2-(2-carboxyethoxy)ethyl]thio]-, bis[[4-(1,1-dimethylethyl)-3-hydroxy-2,6-dimethylphenyl]methyl]ester; Propanoic acid, 3-[[2-(2-carboxyethoxy)ethyl]thio]-,bis[[3-(1,1-dimethylethyl)-4-hydroxy-5-methylphenyl]methyl]ester; Propanoic acid, 3,3′-[1,2-ethanediylbis(thio)]bis-, bis[[3-(1,1-dimethylethyl)-4-hydroxy-5-methylphenyl]methyl]ester; Propanoic acid, 3,3′-thiobis-, bis[[3-(1,1-dimethylethyl)-4-hydroxy-5-methylphenyl]methyl]ester; Acetic acid, 2,2′-thiobis-,bis[[3-(1,1-dimethylethyl)-4-hydroxy-5-methylphenyl]methyl]ester; Succinic acid, 2-[(2-carboxyethyl)thio]-, tris[[4-(1,1-dimethylethyl)-3-hydroxy-2,6-dimethylphenyl]methyl]ester; Succinic acid, 2,2′-thiobis-, tetrakis[[4-(1,1-dimethylethyl)-3-hydroxy-2,6-dimethylphenyl]methyl]ester; Benzoic acid, 3-(1,1-dimethylethyl)-4-hydroxy-5-methyl-, thiodi-2,1-ethanediyl ester; Benzoic acid, 3,5-bis(1,1-dimethylethyl)-4-hydroxy-, thiodi-2,1-ethanediyl ester; Succinic acid, 2-[(2-carbooctyloxyethyl)thio]-, bis[[4-(1,1-dimethylethyl)-3-hydroxy-2,6-dimethylphenyl]methyl]ester; Benzenepropionic acid, 3-(1,1-dimethylethyl)-α-[3-(1,1-dimethylethyl)-4-hydroxy-5-methylphenyl]-4-hydroxy-α,5-dimethyl-, thiodi-2,1-ethanediyl ester; Benzenepropionic acid, 3-(1,1-dimethylethyl)-α-[3-(1,1-dimethylethyl)-4-hydroxy-5-methylphenyl]-4-hydroxy-5-methyl-, thiodi-2,1-ethanediyl ester; Benzenepropionic acid, 3-(1,1-dimethylethyl)-α-[3-(1,1-dimethylethyl)-4-hydroxyphenyl]-4-hydroxy-α-methyl-, thiodi-2,1-ethanediyl ester; Benzeneacetic acid, 3-(1,1-dimethylethyl)-α-[3-(1,1-dimethylethyl)-4-hydroxyphenyl]-4-hydroxy, thiodi-2,1-ethanediyl ester; Benzeneacetic acid, 3-(1,1-dimethylethyl)-α-[3-(1,1-dimethylethyl)-4-hydroxyphenyl]-4-hydroxy-α-methyl-,thiodi-2,1-ethanediyl ester; Benzeneacetic acid, 3-(1,1-dimethylethyl)-α-[3-(1,1-dimethylethyl)-4-hydroxyphenyl]-4-hydroxy, thiodi-2,1-ethanediyl ester; Benzeneacetic acid, 3-(1,1-dimethylethyl)-α-[3-(1,1-dimethylethyl)-4-hydroxy-5-methylphenyl]-4-hydroxy-α,5-dimethyl-, thiodi-2,1-ethanediyl ester; Benzeneacetic acid, 3-(1,1-dimethylethyl)-α-[3-(1,1-dimethylethyl)-4-hydroxy-5-methylphenyl]-4-hydroxy-5-methyl-, thiodi-2,1-ethanediyl ester; Propenoic acid, 2-[[4-(1,1-dimethylethyl)-3-hydroxy-2,6-dimethylbenzyl]thio]ethyl ester, homopolymer; Propenoic acid, 2-[(3-tert-butyl-4-hydroxy-5-methylbenzyl)thio]ethyl ester, homopolymer, Propenoic acid, 2-methyl-, 2-[[4-(1,1-dimethylethyl)-3-hydroxy-2,6-dimethylbenzyl]thio]ethyl ester, homopolymer; Propenoic acid, 2-methyl-, 2-[(3-tert-butyl-4-hydroxy-5-methylbenzyl)thio]ethyl ester, homopolymer; Acetic acid, 2-octadecylthio-, 3-(1,1-dimethylethyl)-4-hydroxy-5-methylphenyl]methyl ester; Acetic acid, 2-hexadecylthio-, 3-(1,1-dimethylethyl)-4-hydroxy-5-methylphenyl]methyl ester; Acetic acid, 2-tetradecylthio-, 3-(1,1-dimethylethyl)-4-hydroxy-5-methylphenyl]methyl ester; Acetic acid, 2-dodecylthio-, 3-(1,1-dimethylethyl)-4-hydroxy-5-methylphenyl]methyl ester; Acetic acid, 2-octadecylthio-, [4-(1,1-dimethylethyl)-3-hydroxy-2,6-dimethylphenyl]methyl ester; Acetic acid, 2-hexadecylthio-, [4-(1,1-dimethylethyl)-3-hydroxy-2,6-dimethylphenyl]methyl ester; Acetic acid, 2-tetradecylthio-, [4-(1,1-dimethylethyl)-3-hydroxy-2,6-dimethylphenyl]methyl ester; Acetic acid, 2-dodecylthio-, [4-(1,1-dimethylethyl)-3-hydroxy-2,6-dimethylphenyl]methyl ester; and mixtures thereof.

In an embodiment, spandex polymer or prepolymer compositions comprise spandex polymer or prepolymer and a stabilizing amount of a compound of the formula (I) or mixture of compounds of the formula (I). It will be understood that reference herein to a compound of the formula (I) is a reference to various individual types of compounds of the formula (I) (such as compounds of the formulas (Ia), (Ib), (Ic), (Id), (Ie), (If), and/or (Ig)), a reference to various individual compounds of the formula (I) (such as AO-1, AO-2, AO-3, AO-7, AO-8, and AO-9), and a reference to various mixtures of one of more compounds represented by formula (I). For any particular spandex polymer or prepolymer composition, the stabilizing amount of the compound of formula (I) may be determined by routine experimentation, guided by the disclosure provided herein. For example, a stabilizing amount of the compounds of formula (I) is often in the range of about 0.01% to about 10% by weight based on the total weight of the stabilized spandex polymer or prepolymer composition, although larger or smaller amounts may be useful in particular situations. In preferred embodiments, the stabilizing amount of the compound of formulas (I) is in the range of about 0.05% to about 5% by weight based on the total weight of the of the stabilized spandex polymer or prepolymer composition.

Compounds of the formula (I) may be prepared using methods known to those skilled in the art or variations of those methods (e.g., methods adapted to produce the various novel compounds of the formula (I)) using routine experimentation guided by the disclosure provided herein. Examples of methods that may be used or adapted to prepare compounds of the formula (I) include those disclosed in U.S. Pat. Nos. 5,892,097; 5,510,402; 3,810,929; 3,923,869; 2,416,052; all of which are hereby incorporated by reference in their entireties and particularly for the purpose of describing methods which may be used or adapted to prepare compounds of the formula (I).

For example, in an embodiment, compounds of the formula (Id), such as (AO-2), can be prepared by reacting an alcohol of formula (Xa) with a carboxylic acid, carboxylic acid chloride or carboxylic acid ester of formula (XIa) in the presence of an appropriate acid or base catalyst or coreagent, and alternatively in the presence of an appropriate solvent, such as toluene or xylene, where the variables in formulas (Xa) and (Ma) are the same as defined above for the corresponding product of the formula (Id) and R⁵ is an appropriate leaving group (such as —OH, —Cl or —O-alkyl):

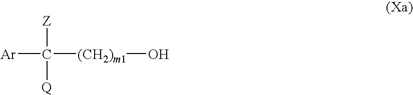

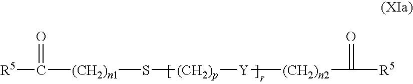

Alternatively, compounds of the formula (Id) can be prepared by reacting an appropriately halogenated compound of the formula (Xb) with a carboxylic acid salt of the formula (XIb), where the variables in formulas (Xb) and (XIb) are the same as defined above for the corresponding product of the formula (Id), X is a halogen such as Cl or Br, and M is H, or a metal or ammonium ion:

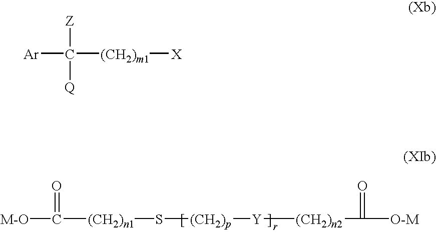

The salt of the formula (XIb) may be used in an isolated form, or prepared in situ from the corresponding carboxylic acid using an appropriate base.

In an embodiment, compounds of formula (Ie), such as AO-3 and AO-8, can be prepared by reacting an appropriately halogenated compound of the formula (Xb) with a thiol of the formula (XIIa), either with or without a suitable organic solvent, such as toluene or xylene, and with or without a suitable organic base, such as triethylamine, or inorganic base, such as potassium carbonate, where the variables in formulas (Xb) and (XIIa) are the same as defined above for the corresponding product of the formula (Ie).

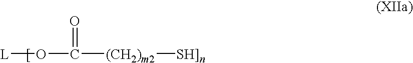

In an alternative embodiment, the compounds of the formula (Ie) can also be prepared by reaction of an n-functional alcohol of the formula (XMa) with a carboxylic acid, carboxylic acid chloride or carboxylic acid ester of the formula (XIVa), where the variables in formulas (XIIIa) and (IVa) are the same as defined above for the corresponding product of the formula (Ie) and R⁵ is an appropriate leaving group (such as —OH, —Cl or —O— alkyl):

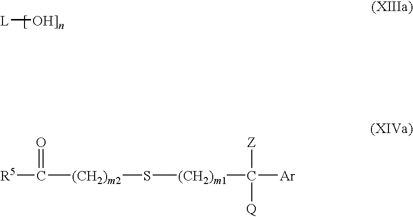

In an embodiment, compounds of formula (If) can be prepared by reacting an alcohol of the formula (XVa) with an n-functional carboxylic acid, carboxylic acid chloride or carboxylic acid ester of the formula (XVIa), where the variables in formulas (XVa) and (XVIa) are the same as defined above for the corresponding product of the formula (If) and R⁵ is an appropriate leaving group (such as —OH, —Cl or O-alkyl):

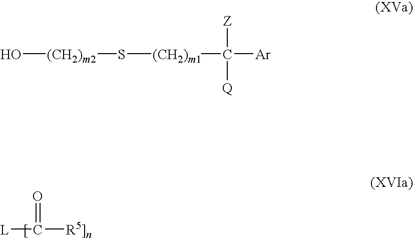

In an alternative embodiment, compounds of the formula (If) may be prepared by reacting an appropriately halogenated compound of the formula (XVIIa) with an n-functional carboxylic acid salt of the formula (XVIIIa) where the variables in formulas (XVIIa) and (XVIIIa) are the same as defined above for the corresponding product of the formula (If), X is a halogen such as Cl or Br, and M is H, or a metal or ammonium ion:

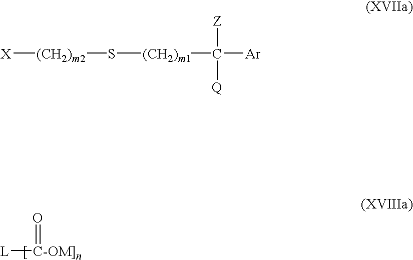

The salt of the formula (XVIIIa) may be used in an isolated form, or prepared in situ from the corresponding carboxylic acid using an appropriate base.

Additional embodiments provide the compounds AO-2, AO-3 and AO-9. Methods for making AO-2, AO-3 and AO-9 are described in the examples below. The compounds AO-2, AO-3 and AO-9 are useful stabilizers for a variety of polymers, including spandex polymer or prepolymer. An embodiment provides a composition that comprises a polymer and AO-2; another embodiment provides a composition that comprises a polymer and AO-3; and another embodiment provides a composition that comprises a polymer and AO-9. The amount of AO-2, AO-3 and AO-9, respectively, in each of these compositions is preferably an amount that is effective to stabilize the polymer. Preferably, the polymer in each of these compositions is spandex polymer or prepolymer.

It will be understood by those skilled in the art that spandex may be prepared by various methods known to those skilled in the art, such as by reaction spinning, melt spinning, dry spinning, or wet-spinning a solution containing the spandex polymer either into a column filled with a hot, inert gas such as air, nitrogen, or steam, or into an aqueous bath to remove the solvent. Subsequently, the resulting spandex fiber may then be wound on a cylindrical core to form a spandex supply package.

An embodiment provides a method for making a stabilized spandex polymer or prepolymer composition, comprising intermixing the spandex polymer or prepolymer and a stabilizing amount of one or more compounds of the formula (I), and optionally by further intermixing an effective amount of a co-additive as described herein, in any order. Such intermixing may be carried out in various ways, e.g., by dissolving or slurrying the compound(s) of formula (I) in a carrier or solvent to form a stabilizing composition and applying the stabilizing composition to the spandex, by applying a liquid form (e.g., melt) of one or more compounds of the formula (I) directly to the spandex, by forming the spandex in the presence of the one or more compounds of the formula (I), and/or by forming one or more compounds of the formula (I) in the presence of the spandex. One or more compounds of the formula (I) can be intermixed with the spandex polymer or prepolymer at any stage of manufacture, e.g., during spandex manufacture and/or to the resulting spandex product (such as fiber or fabric). In an embodiment, a stabilizing composition is formed by preparing a mixture comprising one or more compounds of formula (I) in a solvent such as dimethylacetamide (DMAc). Optionally, other additives and/or viscosity enhancers may be added to the mixture. The stabilizing composition is then mixed into the spandex spinning solution prior to spinning. Separate mixtures may also be used for the various additives but it is not necessary to do so.

### Co-Additives

In an embodiment, a stabilized spandex polymer or prepolymer composition comprises spandex polymer or prepolymer, one or more stabilizers of the formula (I), and one or more co-additives, such as the co-additives described below. Mixtures are specifically contemplated, both within each type of co-additive and among different types. The co-additives may be obtained from commercial sources or synthesized using known synthetic methods, and intermixed with the spandex polymer or prepolymer in a manner generally known to those skilled in the art or in the general manner described above, using routine experimentation as informed by the knowledge of those skilled in the art guided by the disclosure provided herein. The co-additives are preferably included in amounts that are effective to stabilize or further stabilize the stabilized spandex polymer or prepolymer composition. Depending on the co-additive, effective amounts tend to be in the range of from about 0.01 to about 10%, preferably from about 0.05% to about 5%, and more preferably from about 0.1 to about 2%, by weight based on the weight of the stabilized spandex polymer or prepolymer composition.

### 1. Antioxidants

In an embodiment, a stabilized spandex polymer or prepolymer composition comprises spandex polymer or prepolymer, a stabilizing amount of a compound of formula (I), and a substantially sulfur-free, hindered phenol antioxidants, preferably in an amount that is effective to stabilize or further stabilize the stabilized spandex composition. In an embodiment, the sulfur-free, hindered phenol antioxidants may be added in an amount from 0.01 to, 10%, preferably from 0.05 to 5%, and more preferably from 0.1 to 2%, based on the weight of the stabilized spandex polymer or prepolymer composition. In another embodiment, the combined amount of the stabilizer of formula (I) and the sulfur-free, hindered phenol antioxidant is less than about 2% based on the total weight of the stabilized spandex polymer or prepolymer composition. In an embodiment, the stabilizer of formulas (I) and the sulfur-free, hindered phenol antioxidant are used in a weight ratio in the range of from about 1:10 to about 10:1.

In a preferred embodiment, the substantially sulfur-free, hindered phenol antioxidant is selected from 1,3,5-tris(4-tert-butyl-3-hydroxy-2,6 dimethylbenzyl)-1,3,5-triazine-2,4,6-(1H,3H,5H)-trione (CYANOX® 1790), triethyleneglycol bis-3-(3-tert-butyl-4-hydroxy-5-methylphenyl)propionate (IRGANOX® 245), 9-bis(2-(3-(3-tert-butyl-4-hydroxy-5-methylphenyl)propionyloxy)-1,1-dimethylethyl)-2,4,8,10-tetraoxaspiro[5.5]undecane 6 (SUMILIZER™ GA-80), 2,6-di-tert-butyl-4-methylphenol, 2-tert-butyl-4,6-di-methylphenol, 2,6-di-tert-butyl-4-ethylphenol, 2,6-di-tert-butyl-4-n-butylphenol, 2,6-di-tert-butyl-4-isobutylphenol, 2,6-dicyclopentyl-4-methylphenol, 2-(Q-methylcyclohexyl)-4,6-dimethylphenol, 2,6-dioctadecyl-4-methylphenol, 2,4,6-tricyclohexylphenol, 2,6-di-tert-butyl-4-meth-oxymethylphenol, ortho-nonylphenol, 2,6-di-nonyl-4-methylphenol, 2,4-dimethyl-6-(1′-methylundec-1′-yl)phenol, 2,4-dimethyl-6-(1′-methylheptadec-1′-yl)phenol, 2,4-dimethyl-6-(1′-methyltridec-1′-yl)phenol, ethylene-1,2-bis(3,3-bis[3-t-butyl-4 hydroxyphenyl]butyrate), 1,1-bis(2-methyl-5-t-butyl-4-hydroxyphenyl) butane, and 1,1,3-tris(2-methyl-5-t-butyl-4-hydroxyphenyl)butane (LOWINOX® CA22 Great Lakes Chemicals).

Examples of preferred substantially sulfur-free, hindered phenol antioxidant include (AO-4), (AO-5), (AO-6), and mixtures thereof:

AO-4—1,3,5-tris(4-tert-butyl-3-hydroxy-2,6 dimethylbenzyl)-1,3,5-triazine-2,4,6-(1H,3H,5H)-trione (CYANOX® 1790):

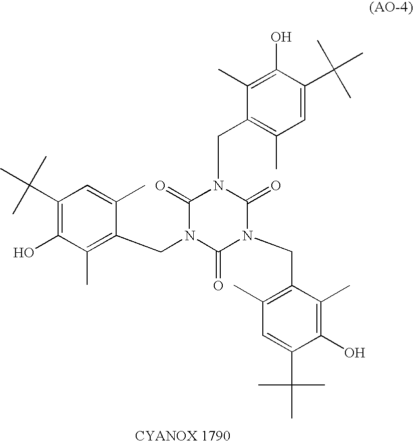

AO-5—triethyleneglycol bis-3-(3-tert-butyl-4-hydroxy-5-methylphenyl) propionate (IRGANOX® 245):

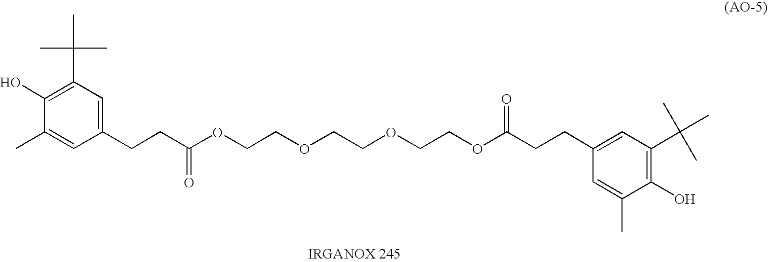

AO-6—9-bis(2-(3-(3-tert-butyl-4-hydroxy-5-methylphenyl)propionyloxy)-1,1-dimethylethyl)-2,4,8,10-tetraoxaspiro[5.5]undecane 6 (SUMILIZER™ GA-80):

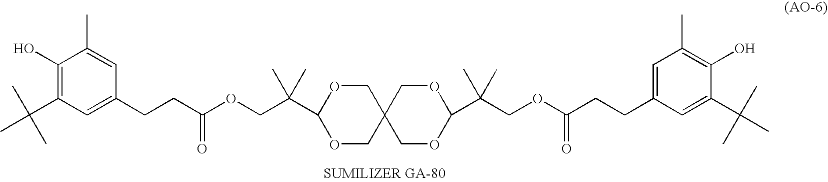

In an embodiment, a stabilized spandex polymer or prepolymer composition comprises spandex polymer or prepolymer, a stabilizer of formula (I), and one or more of the following antioxidants, in an amount that is effective to inhibit oxidation of the spandex:

Alkylated monophenols, for example, 2,6-di-tert-butyl-4-methylphenol, 2-tert-butyl-4,6-di-methylphenol, 2,6-di-tert-butyl-4-ethylphenol, 2,6-di-tert-butyl-4-n-butylphenol, 2,6-di-tert-butyl-4-isobutylphenol, 2,6-dicyclopentyl-4-methylphenol, 2-α-methylcyclohexyl)-4,6-dimethylphenol, 2,6-ioctadecyl-4-methylphenol, 2,4,6-tricyclohexylphenol, 2,6-di-tert-butyl-4-meth-oxymethylphenol. Nonylphenols which are linear or branched in the side chains, for example, 2,6-di-nonyl-4-methylphenol, 2,4-dimethyl-6-(1′-methylundec-1′-yl)phenol, 2,4-dimethyl-6-(1′-methylheptadec-1′-yl)phenol, 2,4-dimethyl-6-(1′-methyltridec-1′-yl)phenol and mixtures thereof.

Alkylthiomethylphenols, for example, 2,4-dioctylthiomethyl-6-tert-butylphenol, 2,4-dioctylthiomethyl-6-methylphenol, 2,4-dioctylthiomethyl-6-ethylphenol, 2,6-di-dodecylthiomethyl-4-nonyl phenol.

Hydroquinone and alkylated hydroquinones, for example, 2,6-di-tert-butyl-4-methoxy-phenol, 2,5-di-tert-butylhydroquinone, 2,5-di-tert-amylhydroquinone, 2,6-diphenyl-4-octade-cyloxyphenol, 2,6-di-tert-butylhydroquinone, 2,5-di-tert-butyl-4-hydroxyanxsole, 3,5-di-tert-butl-4-hydroxyanisole, 3,5-di-tert-butyl-4-hydroxyphenyl stearate, bis(3,5-di-tert-butyl-4-hydroxyphenyl)adipate.

Tocopherols, for example, α-1-tocopherol, β-tocopherol, λ-tocopherol, δ-tocopheroland mixtures thereof (vitamin E).

Hydroxylated thiodiphenyl ethers, for example, 2,2′-thiobis(6-tert-butyl-4-methylphenol), 2,2′-thiobis(4-octylphenol), 4,4′-thiabis(6-tert-butyl-3-methylphenol), 4,4′-thiobis(6-tert-butyl-2-methylphenol), 4,4′-thiobis(3,6-di-sec-amylphenol), 4,4′-bis(2,6-dimethyl-4-hydroxyphenyl)disulfide.

Alkylidenebisphenols, for example, 2,2′-methylenebis(6-tert-butyl-4-methylphenol), 2,2′-methylenebis(6-tert-butyl-4-ethylphenol), 2,2′-methylenebis[4-methyl-6-(α-methylcyclohexyl)phenol], 2,2′-methylenebis(4-methyl-6-cyclohexylphenol), 2,2′-methylenebis(6-nonyl-methylphenol), 2,2′-methylenebis(4,6-di-tert-butylphenol), 2,2′-ethylidenebis(4,6-di-tert-butylphenol), 2,2′-ethylidenebis(6-tert-butyl-4-isobutylphenol), 2,2′-methylenebis[6-(α-methylbenzyl)-4-nonylphenol], 2,2′-methylenebis[6-(α,α-dimethylbenzyl)-4-nonylphenol], 4,4′-methylenebis(2,6-di-tert-butylphenol), 4,4′-methylenebis(6-tert-butyl-2-methylphenol), 1,1-bis(5-tert-butyl-4-hydroxy-2-methylphenyl)butane, 2,6-bis(3-tert-butyl-5-methyl-2-hydroxybenzyl) 4-methylphenol, 1,1,3-tris(5-tert-butyl-4-hydroxy-2-methylphenyl)butane, 1,1-bis(5-tert-butyl-4-hydroxy-2-methylphenyl)-3-n-dodecylmercaptobutane, ethylene glycolbis[3,3-bis(3′-tert-butyl-4′-hydroxyphenyl)butyrate], bis(3-tert-butyl-4-hydroxy-5-methyl-phenyl)dicyclopentadiene, bis[2-(3′-tert-butyl-2′-hydroxy-5′-methylbenzyl)-6-tert-butyl-4-methylphenyl]terephthalate, 1,1-bis-(3,5-dimethyl-2-hydroxyphenyl)butane, 2,2-bis(3,5-di-tert-butyl-4-hydroxyphenyl)propane, 2,2-bis-(5-tert-butyl-4-hydroxy-2-methylphenyl)-4-n-dodecylmercaptobutane, 1,1,5,5-tetra(5-tert-butyl-4-hydroxy-2-methylphenyl)pentane.

Compound that contain O-, N- and S-benzyl groups, for example, 3,5,3′,5′-tetra-tert-butyl-4,4′-dihydroxydi-benzyl ether, octadecyl-4-hydroxy-3,5-dimethylbenzylmercaptoacetate, tridecyl-4-hydroxy-3,5-di-tert-butylbenzylmercaptoacetate, tris(3,5-di-tert-butyl-4 hydroxybenzyl)amine, bis(4 tert-butyl-3-hydroxy-2,6-dimethylbenzyl)dithioterephthalate,-bis(3,5-di-tert-butyl-4-hydroxybenzyl)sulfide, isooctyl-3,5-di-tert-butyl-4-hydroxybenzylmercaptoacetate.

Hydroxybenzylated malonates, for example, dioctadecyl-2,2-bis(3,5-di-tert-butyl-2-hydroxybenzyl)malonate, di-octadecyl-2-(3-tert-butyl-4-hydroxy-5 methylbenzyl)malonate, di-dodecylmercaptoethyl-2,2-bis(3,5-di-tert-butyl-4-hydroxybenzyl)malonate, bis[4-(1,1,3,3-tetramethylbutyl)phenyl]-2,2-bis(3,5-di-tert-butyl-4-hydroxybenzyl)malonate.

Aromatic hydroxybenzyl compounds, for example, 1,3,5-tris(3,5-di-tert-butyl-4-hydroxy-benzyl)-2,4,6-trimethylbenzene, 1,4-bis(3,5-di-tert-butyl-4-hydroxybenzyl) 2,3,5,6-etramethylbenzene, 2,4,6-tris(3,5-di-tert-butyl-hydroxybenzyl)phenol.

Triazine compounds, for example, 2,4-bis(octylmercapto)-6-(3,5-di-tert-butyl-4-hydroxy-anilino)-1,3,5-triazine, 2-octylmercapto-4,6-bis(3,5-ditert-butyl-4-hydroxyanilino)-1,3,5-triazine, 2-octylmercapto-4,6-bis(3,5-di-tert-butyl-4 hydroxyphenoxy)-1,3,5-triazine, 2,4,6-tris-I(3,5-di-tert-butyl-4-hydroxyphenoxy)-1,2,3-triazine, 1,3,5-tris(3,5-di-tert-butyl-4-hydroxybenzyl)isocyanurate, 1,3,5-tris(4-tert-butyl-3-hydroxy-2,6-dimethylbenzyl)isocyanurate, 2,4,6-tris(3,5-di-tert-butyl-4-hydroxyphenylethyl)-1,3,5-triazine, 1,3,5-tris(3,5-di-tert-butyl-4-hydroxylphenylpropionyl)-hexahydro-1,3,5-triazine, 1,3,5-tris(3,5-dicyclohexyl-4 hydroxybenzyl)isocyanurate.

Benzylphosphonates, for example, dimethyl-2,5-di-tert-butyl-4-hydroxybenzylphosphonate, diethyl-3,5-di-tert-butyl-4 hydroxybenzylphosphonate, dioctadecyl-3,5-di-tert-butyl-4-hydroxybenzylphosphonate, dioctadecyl-5-tert-butyl-4-hydroxy-3-methylbenzylphosphonate, the calcium salt of the monoethyl ester of 3,5-di-tert-butyl-4-hydroxybenzylphosphonic acid.

Acylaminophenols, for example 4-hydroxylauranilide, 4-hydroxystearanilide, octyl N-(3,5-di-tert-butyl-4-hydroxyphenyl)carbamate.

Esters of β-(3,5-di-tert-butyl-4-hydroxyphenyl)propionic acid with mono- or polyhydric alcohols, e.g. with methanol, ethanol, n-octanol, i-octanol, octadecanol, 1,6-hexanediol, 1,9-nonanediol, ethylene glycol, 1,2-propanediol, neopentyl glycol, thiodiethylene glycol, diethyleneglycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

Esters of β-(5-tert-butyl-4-hydroxy-3-methylphenyl)propionic acid with mono- or polyhydric alcohols, for example, with methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythiritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane; 3,9-bis[2-{3-(3-tert-butyl-4-hydroxy-5-methylphenyl)propionyloxy}-1,1-dimethylethyl]2,4,8,10-tetraoxaspiro[5.5]undecane.

Esters of α-(3,5-dicyclohexyl-4-hydroxyphenyl)propionic acid with mono- or polyhydric alcohols, e.g. with methanol, ethanol, octanol, octadecanol, 1,6 hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, timethylhexanediol, trimethylolpropane, 4-hydroxymethyl-1-phospha-2,6,7-trioxabicyclo[2.2.2]octane.

Esters of 3,5-di-tert-butyl-4-hydroxyphenyl acetic acid with mono- or polyhydric alcohols, e.g. with methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

Amides of β-(3,5-di-tert-butyl-4-hydroxyphenyl)propionic acid e.g. N,N′-bis(3,5-di-tert-butyl-4 hydroxyphenylpropionyl)hexamethylenediamide, N,N′-bis(3,5-di-tert-butyl-4-hydroxy-phenylpropionyl)trimethylenediamide, N,N′-bis(3,5-di-tert-butyl-4-hydroxyphenylpropionyl)hydrazide, N,N′-bis[2-(3-[3,5-di-tert-butyl-4-hydroxyphenyl]propionyloxy)ethyl]oxamide.

Ascorbic acid (vitamin C).

Aminic antioxidants, for example, N,N′-di-isopropyl-p-phenylenediamine, N,N′-di-sec-butyl-p-phenylenediamine, N,N′-bis(1,4-dimethylpentyl)-p-phenylenediamine, N,N′-bis(1-ethyl-3-methylpentyl)-p-phenylenediamine, N,N′-bis(1-methylheptyl)-p-phenylenediamine, N,N′-dicyclohexyl-p-phenylenediamine, N,N′-diphenyl-p-phenylenediamine, N,N′-bis(2-naphthyl)-p-phenylenediamine, N-isopropyl-N′-phenyl-p-phenylenediamine, N-(1,3 dimethylbutyl)-N′-phenyl-p-phenylenediamine, N-(1-methylheptyl)-N′-phenyl-p phenylenediamine, N-cyclohexyl-N′-phenyl-p-phenylenediamine, 4-(p-toluenesulfamoyl)diphenylamine, N,N′-dimethyl-N,N′-di-sec-butyl-p-phenylenediamine, diphenylamine, N-allyidiphenylamine, 4-isopropoxydiphenylamine, N-phenyl-1-naphthylamine, N-(4-tert-octylphenyl)-1-naphthylamine, N-phenyl-2-naphthylamine, octylated diphenylamine, for example p,p′-di-tert-octyldiphenylamine, 4-n-butylaminophenol, 4-butyrylaminophenol, 4-nonanoylaminophenol, 4-dodecanoylaminophenol, 4-octadecanoylaminophenol, bis(4-methoxyphenyl)amine, 2,6-di-tert-butyl-4-dimethylamino methylphenol, 2,4′-diaminodiphenylmethane, 4,4′-diaminodiphenylmethane, N,N,N′,N-tetra-methyl-4,4′-diaminodiphenylmethane, 1,2-bis[(2-methylphenyl)aminoethane, 1,2 bis(phenylamino)propane, (o-tolyl)biguanide, bis[4-(1′,3′-dimethylbutyl)phenyl]amine, tert octylated N-phenyl-1-naphthylamine, a mixture of mono- and dialkylated tert-butyl/tert octyidiphenylamines, a mixture of mono- and dialkylated nonyldiphenylamines, a mixture of mono- and dialkylated dodecyldiphenylamines, a mixture of mono- and dialkylatedisopropyl/isohexyldiphenylamines, a mixture of mono- and dialkylated tert-butyldiphenylamines, 2,3-dihydro-3,3-dimethyl-4H-1,4-benzothiazine, phenothiazine, a mixture of mono- and dialkylated tert-butyl/tert-octylphenothiazines, a mixture of mono- and dialkylated tert-octylphenothiazines, N-allylphenothiazine, dinonylphenothiazine, mono-1-nonylphenothiazine, a mixture of mono- and dialkylatednonylphenothiazine, N,N,N′,N′ tetraphenyl-1,4-diaminobut-2-ene, a mixture of one of the above disclosed unsubstituted or substituted diphenylamine with one of the above disclosed unsubstituted or substituted phenothiazine.

### 2. NOₓ Stabilizers

In an embodiment, a stabilized spandex polymer or prepolymer composition comprises spandex polymer or prepolymer, a stabilizing amount of a compound of formula (I), and an NOₓ stabilizer in an amount effective to inhibit NOₓ discoloration. In a preferred embodiment, the NOₓ stabilizer is selected from N,N′-(methylenedi-1,4-phenylene) bis[2,2-dimethyl hydrazine carboxamide] (HN-150), a poly(dialkyl aminoethylmethacrylate), and a lauric acid hydrazide reaction product with bisphenol A diglycidyl ether. In a more preferred embodiment, the NOₓ stabilizer is N,N′-(methylenedi-1,4-phenylene)bis[2,2-dimethyl hydrazine carboxamide] (HN-150). The NOₓ stabilizer, in an embodiment, is included in the spandex polymer or prepolymer composition in an amount in the range of 0.1 to 2.0%, preferably 0.3 to 1.5%, by weight based on the weight of the stabilized spandex polymer or prepolymer composition.

### 3. UV Absorbers and Light Stabilizers

In an embodiment, a stabilized spandex polymer or prepolymer composition comprises spandex polymer or prepolymer, a stabilizing amount of a compound of formulas (I), and a UV stabilizer and/or light stabilizer, the UV stabilizer and/or light stabilizer being present in an amount that is effective to inhibit photochemically initiated degradation reactions. Examples of UV stabilizers and/or light stabilizers include:

2-(2′-Hydroxyphenyl)benzotriazoles, for example, 2-(2′-hydroxy-5′-methylphenyl)benzo triazole,2-(3′,5′-di-tert-butyl-2′-hydroxyphenyl)benzotriazole, 2-(5′-tert-butyl-2′-hydroxyphenyl)benzotriazole, 2-(2′-hydroxy-5′-(1,1,3,3-tetramethylbutyl)phenyl)benzotriazole, 2-(3′,5′-di-tert-butyl-2′-hydroxyphenyl)-5-chlorobenzotriazole, 2-(3′-tert-butyl-2′-hydroxy-5′-methylphenyl)-5-chlorobenzotriazole, 2-(3′-sec-butyl-5′-tert-butyl-2′-hydroxyphenyl)benzotriazole, 2-(2′ hydroxy-4′-octyloxyphenyl)benzotriazole, 2-(3′,5′-di-tert-amyl-2′ hydroxyphenyl)benzotriazole, 2-(3′,5′-bis α,α-dimethylbenzyl)-2′-hydroxyphenyl)benzotriazole, 2-(3′-tert-butyl-2′-hydroxy-5′-(2-octyloxycarbonylethyl)phenyl) 5-chlorobenzotriazole, 2-(3′-tert-butyl-5′-[2-(2-ethylhexyloxy)carbonylethyl]-2′ hydroxyphenyl)-5-chlorobenzotriazole, 2-(3′-tert-butyl-2′-hydroxy-5′-(2-methoxycarbonylethyl)phenyl)-5-chlorobenzotriazole, 2-(3′-tert butyl-2′-hydroxy-5′-(2-methoxycarbonylethyl)phenyl)benzotriazole, 2-(3′-tert-butyl-2′-hydroxy-5′-(2-octyloxycarbonyl ethyl)phenyl)benzotriazole, 2-(3′-tert-butyl-5′-[2-(2-ethylhexyloxy)carbonylethyl]-2′-hydroxy phenyl)benzotriazole, 2-(3′-dodecyl-2′-hydroxy-5′-methylphenyl)benzotriazole, 2-(3′-tert-butyl-2′-hydroxy-5′-(2-isooctyloxycarbonylethyl)phenylbenzotriazole, 2,2′-methylenebis[(1,1,3,3-tetramethylbutyl)-6-benzotriazole-2-ylphenol]; the transesterification product of 2 [3′-tert-butyl-5′-(2-methoxycarbonylethyl)-2′-hydroxyphenyl]-2H-benzotriazole with polyethylene glycol 300; [R—CH₂CH₂—COO—CH₂(CH₂)₃]₂ where R=3′-tert-butyl-4′ hydroxy-5′-2H-benzotriazol-2-ylphenyl, 2-[2′-hydroxy-3′-(α,α-dimethylbenzyl)-5′-(1,1,3,3-tetramethylbutyl)phenyl]benzotriazole; 2-[2′-hydroxy-3′-(1,1,3,3-tetraethylbutyl)-5′-(α,α-dimethylbenzyl)phenyl]benzotriazole.

2-Hydroxybenzophenones and Formamidines, for example, the 4-hydroxy, 4-methoxy, 4-octyloxy, 4-decyloxy, 4-dodecyloxy, 4-benzyloxy, 4,2′,4′-trihydroxy and 2′-hydroxy-4,4′-di-methoxy benzophenones; N-alkyl-N,N′-diarylformamidines, for example, benzoic acid, 4-[[(ethylphenylamino)methylene]amino]ethyl ester; 2-propenoic 1 acid, 3-(4-methoxyphenyl)-, 2-ethylhexyl ester [UVINUL® 3088®, BASF]; 2-propenoic acid, 2-cyano-3,3-diphenyl-, ethyl ester [UVINUL® 3035®, BASF]; or 2-propenoic acid, 2-cyano-3,3-diphenyl-, 2-ethylhexyl ester [UV® (D 3039®, BASF].

Esters of substituted and unsubstituted benzoic acids, for example, 4-tert-butylphenyl salicylate, phenyl salicylate, octylphenyl salicylate, dibenzoyl resorcinol, bis(4-tert-butylbenzoyl)resorcinol, benzoylresorcinol, 2,4-di-tert-butylphenyl 3,5-di-tert-butyl-4-hydroxybenzoate, hexadecyl 3,5-di-tert-butyl-4-hydroxybenzoate, octadecyl 3,5-di-tert-butyl-4-hydroxybenzoate, 2-methyl,6-di-tert-butylphenyl 3,5-di-tert-butyl-4-hydroxybenzoate.

Acrylates, for example, ethyl α-cyano-β,β-diphenylacrylate, isooctyl-α-cyano-β,β-diphenylacrylate, methyl α-carbomethoxycinniamate, methyl-α-cyano-methyl-p-methoxycinnamate, butyl α-cyano-β-methyl-p-methoxycinnamate, methyl α-carbomethoxy-p-methoxycinnamate and N-(β-carbomethoxy-β-cyanovinyl)-2-methylindoline.

Sterically hindered amines, for example, bis(2,2,6,6-tetramethyl-4-piperidyl)sebacate, bis(2,2,6,6-tetramethyl-4-piperidyl)succinate, bis(1,2,2,6,6-pentamethyl-4-piperidyl)sebacate, bis(1-octyloxy-2,2,6,6-tetramethyl-4-piperidyl)sebacate, bis(1,2,2,6,6-pentamethyl-piperidyl) n-butyl-3,5-di-tert-butyl-4-hydroxybenzylmalonate, the condensate of 1-(2-hydroxyethyl)-2,2,6,6-tetramethyl-4-hydroxypiperidine and succinic acid, linear or cyclic condensates of N,N′-bis(2,2,6,6-tetramethyl-4-piperidyl)hexamethylenediamine and 4 tert-octylamino-2,6-dichloro-1,3,5-triazine, tris(2,2,6,6-tetramethyl-4-piperidyl)nitrilotriacetate, tetrakis(2,2,6,6-tetramethyl-4-piperidyl)-1,2,3,4-butanetetracarboxylate, 1,1′-(1,2-13ethanedlyl)-bis(3,3,5,5-tetramethylpiperazinone), 4-benzoyl-2,2,6,6-tetramethylpiperidine, 4 stearyloxy-2,2,6,6-tetramethylpiperidine, bis(1,2,2,6,6-pentamethylpiperidyl)-2-n-butyl-2-(2-1 hydroxy-3,5-di-tert-butylbenzyl)malonate, 3-n-octyl-7,7,9,9-tetramethyl-1,3,8 triazaspiro[4.5]decane-2,4-dione, bis(1-octyloxy-2,2,6,6-tetramethylpiperidyl)sebacate, bis(1-octyloxy-2,2,6,6-tetramethylpiperidyl)succinate, linear or cyclic condensates of N,N′ bis(2,2,6,6-tetramethyl-4-piperidyl) hexamethylenediamine and 4-morpholino-2,6-dichloro 1,3,5-triazine, the condensate of 2-chloro-4,6-bis(4-n-butylamino-2,2,6,6-tetramethylpiperidyl)-1,3,5-triazine and 1,2-bis(3-aminopropylamino)ethane, the condensate of 2-chloro-4,6-di-(4-n-butylamino-1,2,2,6,6-pentamethylpiperidyl)-1,3,5-triazine and 1,2 bis(3-aminopropylamino)ethane, 8-acetyl-3-dodecyl-7,7,9,9-tetramethyl-1,3,8 triazaspiro[4.5]decane-2,4-dione, 3-dodecyl-1-2,2,6,6-tetramethyl-4 piperidyl)pyrrolidine 2,5-dione, 3-dodecyl-1-(1,2,2,6,6-pentamethyl-4-piperidyl)pyrrolidine-2,5-dione, a mixture of 4-hexadecyloxy- and 4-stearyloxy-2,2,6,6-tetramethylpiperidine, a condensate of N,N′bis(2,2,6,6-tetramethyl-4-piperidyl)hexamethylenediamine and 4-cyclohexylamino-2,6-dichloro-1,3,5-triazine, a condensate of 1,2-bis(3-aminopropylamino)ethane and 2,4,6-trichloro-1,3,5-triazine as well as 4-butylamino-2,2,6,6-tetramethylpiperidine (CAS Reg. No. [136504-96-6]); a condensate of 1,6-hexanediamine and 2,4,6-trichloro-1,3,5-triazine as well as N,N-dibutylamine and 4-butylamino-2,2,6,6-tetramethylpiperidine (CAS Reg. No. [192268-64 7]); N-(2,2,6,6-tetramethyl-4-piperidyl)-n-dodecylsuccinimide, N-(1,2,2,6,6 pentamethyl-4-piperidyl)-n-dodecylsuccinimide, 2-undecyl-7,7,9,9-tetramethyl-1-oxa-3,8-diaza-4-oxo-spiro[4,5]decane, a reaction product of 7,7,9,9-tetramethyl-2-cycloundecyl-1-oxa 3,8-diaza-4-oxospiro-[4,5]decane and epichlorohydrin, 1,1-bis(1,2,2,6,6-pentamethyl-4-piperidyloxycarbonyl)-2-(4-methoxyphenyl)ethene, N,N′-bis-formyl-N,-bis(2,2,6,6-tetrame thyl-4-piperidyl)hexamethylenediamine, a diester of 4-methoxymethylenemalonic acid with 1,2,2,6,6-pentamethyl-4-hydroxypiperidine, poly[methylpropyl-3-oxy-4-(2,2,6,6-tetramethyl 4-piperidyl)]siloxane, a reaction product of maleic acid anhydride-α-olefin copolymer with 2,2,6,6-tetramethyl-4-aminopiperidine or 1,2,2,6,6-pentamethyl-4-aminopiperidine.

Oxamides, for example, 4,4′-dioctyloxyoxanilide, 2,2′-diethoxyoxanilide, 2,2′-dioctyloxy-5,5′-di-tert-butoxanilide, 2,2′-didodecyloxy-5,5′-di-tert-butoxanilide, 2-ethoxy-2′-ethyloxanilide, N,N-bis(3-dimethylaminopropyl)oxamide, 2-ethoxy-5-tert-butyl-2′-ethoxanilide and its mixture with 2-ethoxy-2′-ethyl-5,4′-di-tert-butoxanilide, mixtures of o- and p-methoxy disubstituted oxanilides, and mixtures of o- and p-ethoxy-disubstituted oxanilides.

2-(2-Hydroxyphenyl)-1,3,5-triazines, for example, 2,4,6-tris(2-hydroxy-4-octyloxyphenyl)-1,3,5-triazine, 2-(2-hydroxy-4-octyloxyphenyl)-4,6-bis(2,4-dimethylphenyl)-1,3,5-triazine, 2-(2-hydroxy-4-iso-octyloxyphenyl)-4,6-bis(2,4-dimethylphenyl)-1,3,5-triazine, 2-(2,4-dihydroxyphenyl)-4,6-bis(2,4-dimethylphenyl)-1,3,5-triazine,2,4-bis(2-hydroxy-4-propyl-oxyphenyl)-6-(2,4-dimethylphenyl)-1,3,5-triazine, 2-(2-hydroxy-4-octyloxyphenyl)-4,6-bis(4-methylphenyl)-1,3,5-triazine, 2-(2-hydroxy-4-dodecyloxyphenyl)-4,6-bis(2,4-dimethylphenyl)-1,3,5-triazine, 2-(2-hydroxy-4-tridecyloxyphenyl)-4,6-bis(2,4-dimethylphenyl)-1,3,5-triazine, 2-[2-hydroxy-4-(2 hydroxy-3-butyloxypropoxy)phenyl]-4,6-bis(2,4-dimethyl)-1,3,5-triazine, 2-[2-hydroxy-4-(2-hydroxy-3-octyloxypropyloxyphenyl]-4,6-bis(2,4-dimethyl)-1,3,5-triazine, 2[4-(dodecyloxy/tridecyloxy-2-hydroxypropoxy)-2-hydroxyphenyl]-4,6-bis(2,4-dimethylphenyl)-1,3,5-triazine, 2-[2-hydroxy-4-(2-hydroxy-3-dodecyloxypropoxy)phenyl]-4,6-bis(2,4-dimethyl phenyl)-1,3,5-triazine, 2-(2-hydroxy-4-hexylaxy)phenyl-4,6-diphenyl-1,3,5-triazine, 2-(2-hydroxy-4-methoxyphenyl)-4,6-diphenyl-1,3,5-triazine, 2,4,6-tris[2-hydroxy-4-(3-butoxy-2-hydroxypropoxy)phenyl]-1,3,5-triazine, 2-(2-hydroxyphenyl)-4-(4-methoxyphenyl)-6-phenyl 1,3,5-triazine, 2-{2-hydroxy-4-[3-(2-ethylhexyl-1-oxy)-2-hydroxypropyloxy]phenyl}-4,6-bis(2,4-dimethylphenyl)-1,3,5-triazine.

### 4. Other Co-Additives

In an embodiment, a stabilized spandex polymer or prepolymer composition comprises spandex polymer or prepolymer, a stabilizing amount of a compound of formula (I), and a stabilizing amount of one or more of the following compounds:

### Phosphites and Phosphonites

For example, triphenyl phosphite, diphenylalkyl phosphites, phenydialkyl phosphites, tris(nonylphenyl)phosphite, trilauryl phosphite, trioctadecyl phosphite, distearylpenteerythritol diphosphite, tris(2,4-di-tert-butylphenyl)phosphite, diisodecyl pentaerythritol diphosphite, bis(2,4-di-tert-butylphenyl)pentaerythritol diphosphite, bis(2,4-di-cumylphenyl)pentaerythritol diphosphite, bis(2,6-di-tert-butyl-4-methylphenyl)pentaerythritol diphosphite, diisodecyloxypentaerythritol diphosphite, bis(2,4-di-tert-butyl-6-methylphenyl)-pentaerythritol diphosphite, bis(2,4,6-tris(tert-butylphenyl)pentaerythritol diphosphite, tristearylsorbitol triphosphite, tetrakis(2,4-di-tert-butylphenyl) 4,4′-biphenylenediphosphonite, 6-isooctyloxy-2,4,8,10-tetra-tert-butyl-12H-d ibenz[d,g]-1,3,2-dioxaphosphocin, bis(2,4-di-tert-butyl-6-methylphenyl)methyl phosphite, bis(2,4-di-tert-butyl-6-methylphenyl)ethyl phosphite, 6-fluoro-2,4,8,10-tetra-tert-butyl-1 2-methyl-dibenz[d,g]-1,3,2-dioxaphosphocin, 2,2′,2″-nitrilo-I[triethyltris(3,3′,5,5′-tetra-tert-butyl-1,1′-biphenyl-2,2′-diyl)phosphite], 2-ethylhexyl(3,3′,5,5′-te-15tra-tert-butyl-1,1′-biphenyl-2,2′-dlyl)phosphite, 5-butyl-5-ethyl-2-(2,4,6-tri-tert-butylphenoxy)-1,3,2-dioxaphosphirane.

### Hydroxylamines

For example, N,N-dibenzylhydroxylamine, N,N-diethylhydroxylamine, N,N-dioctylhydroxylamine, N,N-dilaurylhydroxylamine, N,N-ditetradecylhydroxylamine, N,N-dihexadecylhydroxylamine, N,N-dioctadecylhydroxylamine, N-hexadecyl-N-octadecylhydroxylamine, N-heptadecyl-N-octadecylhydroxylamine, N,N-dialkylhydroxylamine derived from hydrogenated tallow amine.

### Nitrones

For example N-benzyl-alpha-phenylnitrone, N-ethyl-alpha-methylnitrone, N-octyl-alpha-heptylnitrone, N-lauryl-alpha-undecylnitrone, N-tetradecyl-alpha-tridecylnitrone, N-hexadecyl-alpha-pentadecylnitrone, N-octadecyl-alpha-heptadecylnitrone, N-hexadecyl-al-pha-heptadecylnitrone, N-ocatadecyl-alpha-pentadecylnitrone, N-heptadecyl-alpha-hepta-decylnitrone, N-octadecyl-alpha-hexadecylnitrone, nitrone derived from N,N-dialkylhydroxyl-amine derived from hydrogenated tallow amine.

Thiosynergists For example, dilauryl thiodipropionate or distearylthiodipropionate.

### Peroxide Scavengers

For example esters of β-thiodipropionic acid, for example the lauryl, stearyl, myristyl or tridecyl esters, mercaptobenzimidazole or the zinc salt of 2-mercapto-benzimidazole, zincdibutyldithiocarbamate, dioctadecyl disulfide, pentaerythritol tetrakis(β-dodecylmercapto)propionate

### Basic Co-Stabilizers

For example melamine, polyvinylpyrrolidone, dicyandiamide, triallyl cyanurate, urea derivatives, hydrazine derivatives, amines, polyamides, polyurethanes, alkali metal salts and alkaline earth metal salts of higher fatty acids, for example calcium stearate, zinc stearate, magnesium behenate, magnesium stearate, sodium ricinoleate and potassium palmitate, antimony pyrocatecholate or zinc pyrocatecholate.

### Anti-Chlor Agents

For example calcium carbonate, silicates, glass fibers, glass bulbs, asbestos, talc, kaolin, mica, barium sulfate, metal oxides and hydroxides, carbon black, graphite, wood flour and flours or fibers of other natural products, synthetic fibers.

### Benzofuranones and Indolinones

For example, those disclosed in U.S. Pat. Nos. 4,325,863; 4,338,244; 5,175,312; 5,216,052; and 5,252,643; or 3-[4-(2-acetoxyethoxy)phenyl]-5,7-di-tert-butylbenzofuran-2-one, 5,7-di-tert-butyl-3-[4-(2-stearoyloxyethoxy)phenyl]benzofuran-2-one,3,3′-bis[5,7-di-tert-butyl-3-(4-[2-hydroxyethoxy]phenyl)benzofuran-2-one], 5,7-di-tert-butyl-3-(4 ethoxyphenyl)benzofuran-2-one, 3-(4-acetoxy-3,5-dimethylphenyl)-5,7-di-tert-butylbenzofuran-2-one, 3-(3,5-dimethyl-4-pivaloyloxyphenyl)-5,7-di-tert-butylbenzofuran-2-one, 3-(3,4-dimethylphenyl)-5,7-di-tert-butylbenzofuran-2-one, 3-(2,3-dimethylphenyl)-5,7-di-tert-butylbenzofuran-2-one or 3-(2-acetyl-5-isooctylphenyl)-5-isooctylbenzofuran-2-one.

### Other Additives

For example, plasticizers, lubricants, emulsifiers, pigments, rheology additives, catalysts, flow-control agents, optical brighteners, flameproofing agents, antistatic agents and blowing agents.

### EXAMPLES

The following examples are provided to assist one skilled in the art further understand embodiments of the present invention. These examples are intended for illustration purposes and are not to be construed as limiting the scope of the present invention.

The chemical compounds utilized in these examples are summarized below:

The compound of formula (AO-1)-3,3′-thiobispropanoic acid, bis[[4-(1,1-dimethylethyl)-3-hydroxy-2,6-dimethylphenyl]methyl]ester. The compound of formula (AO-1) may be made from 3-(chloromethyl)-6-(1,1-dimethylethyl)-2,4-dimethylphenol as described by U.S. Pat. No. 3,923,869, which is hereby incorporated by reference in its entirely and particularly for the purpose of describing (AO-1) and methods for its preparation. The compound of formula (AO-79) is prepared in a similar manner to that of the formula (AO-1), except that the 2,2′-dimethylthiopropionic acid salt is used instead of the thiodipropionic acid salt.

The compound of formula (AO-2) is prepared according to the following method. Approximately 4.0 g (16.5 mmol) of 3,3′-[1,2-Ethanediylbis(thio)]bispropanoic acid, prepared according to U.S. Pat. No. 2,416,052, is charged to a 250 mL round-bottomed flask equipped with a magnetic stir bar. Approximately 25 mL toluene is charged to the flask and the mixture is magnetically stirred. Approximately 5.15 g (33.8 mmol) of 1,8-Diazabicyclo[5.4.0]undec-7-ene is added dropwise, causing a gum to form. Approximately 25 mL methylene chloride is then added, which dissolves most of the gum. To this mixture is added approximately 7.66 g (33.8 mmol) 3-(chloromethyl)-6-(1,1-dimethylethyl)-2,4-dimethylphenol), followed by approximately 25 mL additional toluene. The mixture is heated to reflux in an oil bath for approximately 20 hours, then cooled to room temperature. After dilution with hexane, the mixture is washed approximately with 2×200 mL of water. Methylene chloride is added to the organic phase to reduce cloudiness, and the combined aqueous layers are washed with methylene chloride. The combined organic layers are rotary evaporated, giving approximately 10.33 g of a sticky red glass. Flash chromatography using approximately 3% ethyl acetate/methylene chloride yields approximately 5.03 g of a sticky colorless glass. Crystallization of approximately 3.5 g of this glass from approximately 10% ethyl acetate/hexane provides approximately 2.7 g of The compound of formula (AO-2) as a white solid.

The compound of formula (AO-3) is prepared as follows: About 1.76 g (4.4 mmol) of 3-mercaptopropanoic acid, 2-ethyl-2-[[3-mercapto-1-oxopropoxy]methyl]-1,3-propanediyl ester, a commercially available compound, is charged to a 100 mL round-bottomed flask equipped with a magnetic stir bar, followed by about 3.0 g (13.2 mmol) 3-(chloromethyl)-6-(1,1-dimethylethyl)-2,4-dimethylphenol and about 15 mL toluene. The mixture is magnetically stirred and about 2.01 g (13.2 mmol) 1,8-diazabicyclo[5.4.0]undec-7-ene is added dropwise, causing an oil to form. About 9 mL methylene chloride is then added, dissolving most of the oil. The mixture is heated to reflux in an oil bath for about 20 hours, then cooled to room temperature. The mixture is diluted with about 200 mL of methylene chloride, then washed approximately with 2×150 mL of water. After drying (MgSO₄) and filtration, rotary evaporation provides about 4.4 g of a reddish glass. Flash chromatography using about 4% ethyl acetate/methylene chloride yields about 2.9 g of the compound of formula (AO-3) as a white foam.

The compound of formula (AO-4)-1,3,5-Tris(4-tert-butyl-3-hydroxy-2,6 dimethylbenzyl)-1,3,5-triazine-2,4,6-(1H,3H,5H)-trione. The compound of formula (AO-4) is commercially available as CYANOX® 1790.

The compound of formula (AO-5)-Triethyleneglycol bis-3-(3-tert-butyl-4-hydroxy-5-methylphenyl)propionate. The compound of formula (AO-5) is commercially available as IRGANOX® 245.

The compound of formula (AO-6)-3,9-bis(2-(3-(3-tert-butyl-4-hydroxy-5-methylphenyl)propionyloxy)-1,1-dimethylethyl)-2,4,8,10-tetraoxaspiro[5.5]undecane. The compound of formula (AO-6) is commercially available as SUMILIZER™ GA-80.

The compound of formula (AO-7) is prepared as follows: Approximately 5.0 g (20 mmol) of benzenepropanoic acid, 3-(1,1-dimethylethyl)-4-hydroxy-5-methyl-, methyl ester is dissolved in approximately 100 mL of toluene in a 250 mL three-necked, round-bottomed flask equipped with a magnetic stir bar and fitted with a Dean-Stark trap and condenser. The mixture is heated to reflux and approximately 30 mL of toluene was drained off. After cooling, approximately 1.22 g (10 mmol) of 2,2′-thiobisethanol is added as a suspension in approximately 5 mL of toluene, followed by approximately 0.11 g (0.4 mmol) of titanium (IV) isopropoxide via syringe. The mixture is heated for approximately 10 hours, over which time toluene is removed slowly. The mixture is cooled to room temperature, and approximately 25 mL methylene chloride added, followed by approximately 0.1 mL of water. The mixture is stirred at room temperature overnight, filtered, and rotary evaporated to yield approximately 5.5 g of a hazy, colorless oil. Flash chromatography using approximately 35% ethyl acetate/hexane provides approximately 2.4 g of the compound of formula (AO-7) in the form of a clear, colorless and viscous liquid.

Stabilized spandex polymer or prepolymer compositions utilizing the AO stabilizers described above are prepared as follows: A spandex pre-polymer solution is prepared by mixing polytetramethylene ether glycol (PTMEG), 1,1′-methylenebis(4-isocyanatobenzene) (MDI), 1,2-diaminopropane, ethylenediamine chain extenders, and diethylamine chain terminator in DMAc solvent. The spandex pre-polymer solution is diluted with DMAc to approximately 25 wt. % solids and approximately 50 gram portions are placed in jars. The AO stabilizers are dissolved into approximately equal portions of the pre-polymer on the weight of resin solid. Spandex films of each composition are cast onto glass plates using a draw down bar and dried for approximately 3 minutes at approximately 200° C. Control samples of spandex containing no antioxidant are similarly prepared.

The cast film compositions of the control and stabilized spandex are tested for color stability in the as-processed state and during oven aging. Aging was performed at approximately 200° C. and 120° C. and during NOₓ exposure at approximately 60° C. The NOₓ exposure test is conducted according to the American Association of Textile Chemists and Colorists (AATCC) Test Method 23-2004, “Colorfastness to Burnt Gas Fumes”.

The cast films of the control and stabilized spandex are mounted onto cardboard holders and measured for initial color using a MacBeth Color-Eye 7000 colorimeter. The measurements are summarized in the examples and tables below. The tables present the color of stabilized spandex polymer or prepolymer compositions, as measured by their yellowness index (YI), and the corresponding change in the yellowness index, on a percentage basis (Δ%), compared to the control.

### Examples 1-13

The effect of the stabilizer on the color of stabilized spandex polymer or prepolymer compositions during oven aging at 200° C. at time intervals of approximately 5, 10, 15, and 25 minutes is determined. The color of spandex polymer or prepolymer compositions stabilized with AO-1, AO-2, and AO-3, as characterized by yellowness index, are compared to that of spandex polymer or prepolymer compositions stabilized with traditional stabilizers AO-4, AO-5, and AO-6. Additionally, the color of spandex polymer or prepolymer compositions stabilized with combinations of AO-1, AO-2, and AO-3 with AO-4, AO-5, and AO-6 are evaluated. The results of these evaluations are presented in Table 3 below.

TABLE 3
OVEN AGING AT 200° C. - YELLOWNESS INDEX VERSUS AGING TIME

| | | Aging Time (minutes) | | | | | | | | | |
|---|---|---|---|---|---|---|---|---|---|---|---|
| | Sample | 0 | | 5 | | 10 | | 15 | | 25 | |
| No. | Wt % | YI | Δ (%) | YI | Δ (%) | YI | Δ (%) | YI | Δ (%) | YI | Δ (%) |
| 1C | Control | 3.5 | 0.0 | 9.7 | 0.0 | 20.7 | 0.0 | 44.1 | 0.0 | 78.0 | 0.0 |
| 2C | 1% AO-4 | 3.5 | 0.0 | 7.1 | −26.8 | 9.7 | −53.1 | 13.4 | −69.6 | 22.9 | −70.6 |
| 3C | 1% AO-5 | 3.7 | 5.7 | 7.4 | −23.7 | 9.9 | −52.2 | 13.4 | −69.6 | 23.4 | −70.0 |
| 4C | 1% AO-6 | 3.5 | 0.0 | 7.4 | −23.7 | 10.4 | −49.8 | 15.4 | −65.1 | 31.9 | −59.1 |
| 5 | 1% AO-1 | 3.0 | −14.3 | 4.9 | −49.5 | 5.9 | −71.5 | 6.8 | −84.6 | 9.0 | −88.5 |
| 6 | 1% AO-2 | 3.0 | −14.3 | 4.6 | −52.6 | 5.7 | −72.5 | 6.6 | −85.0 | 8.3 | −89.4 |
| 7 | 1% AO-3 | 3.3 | −5.7 | 5.1 | −47.4 | 5.8 | −72.0 | 6.6 | −85.0 | 7.9 | −89.9 |
| 8 | AO-1, AO-4 | 2.9 | −17.1 | 4.5 | −53.6 | 5.5 | −73.4 | 6.6 | −85.0 | 11.4 | −85.4 |
| | 0.5% each | | | | | | | | | | |
| 9 | AO-1, AO-5 | 2.5 | −28.6 | 5.0 | −48.5 | 6.4 | −69.1 | 8.1 | −81.6 | 12.7 | −83.7 |
| | 0.5% each | | | | | | | | | | |
| 10 | AO-1, AO-6 | 3.0 | −14.3 | 5.0 | −48.5 | 6.1 | −70.5 | 7.4 | −83.2 | 11.3 | −85.5 |
| | 0.5% each | | | | | | | | | | |
| 11 | AO-2, AO-4 | 2.9 | −17.1 | 4.5 | −53.6 | 5.2 | −74.9 | 5.8 | −86.8 | 7.3 | −90.6 |
| | 0.5% each | | | | | | | | | | |
| 12 | AO-3, AO-4 | 3.1 | −11.4 | 4.6 | −52.6 | 5.5 | −73.4 | 6.3 | −85.7 | 7.7 | −90.1 |
| | 0.5% each | | | | | | | | | | |
| 13 | AO-1, AO-4 | 2.9 | −17.1 | 5.0 | −48.5 | 6.0 | −71.0 | 6.9 | −84.4 | 10.5 | −86.5 |
| | (0.375% each) and | | | | | | | | | | |
| | 0.25% HN-150 | | | | | | | | | | |

The 200° C. oven aging data presented in Table 3 demonstrate that stabilized spandex polymer or prepolymer compositions using AO-1, AO-2, and AO-3 stabilizers (Examples 5-7) exhibit consistently lower values of yellowness index compared to spandex polymer or prepolymer stabilized with traditional stabilizers AO-4, AO-5, and AO-6 (Examples 2C-4C) under the conditions examined. The yellowness index of spandex polymer or prepolymer compositions stabilized with AO-1, AO-2, and AO-3 is found to range from approximately 6-14% less than the control in the as-processed condition to approximately 90% less than the control after aging for 25 minutes. In contrast, the yellowness index of spandex polymer or prepolymer compositions stabilized with AO-4, AO-5, and AO-6 are found to range from approximately 0-6% less than the control in the as-processed condition to approximately 59-71% less than the control after 25 minutes. Additionally, compared to the control, spandex polymer or prepolymer compositions using combinations of AO-1 with AO-4, AO-5, and AO-6 (Examples 8-10, 13) and combinations of AO-2 and AO-3 with AO-4 (Examples 11-12) exhibit yellowness index values that are comparable to, and in many cases lower than, the individual antioxidants, particularly AO-4, AO-5, and AO-6. In Examples 8-13, the yellowness index is found to range from approximately 11-29% less than the control in the as-processed condition and approximately 87-91% less than the control after 25 minutes.

### Examples 14-19

The effect of the stabilizer on the color of stabilized spandex polymer or prepolymer compositions during oven aging at 200° C. at time intervals of approximately 5, 10, 15, and 20 minutes is determined. The color of spandex polymer or prepolymer compositions stabilized with AO-1 and AO-7 are compared to spandex polymer or prepolymer compositions stabilized with traditional stabilizers AO-4 and AO-5. Additionally, the color of a spandex polymer or prepolymer composition stabilized with a combination of AO-1 with AO-4 is evaluated. The results of these evaluations are presented in Table 4 below.

TABLE 4
OVEN AGING AT 200° C. - YELLOWNESS INDEX VERSUS AGING TIME

| | | Aging Time (minutes) | | | | | | | | | |
|---|---|---|---|---|---|---|---|---|---|---|---|
| | Sample | 0 | | 5 | | 10 | | 15 | | 20 | |
| No. | Wt % | YI | Δ (%) | YI | Δ (%) | YI | Δ (%) | YI | Δ (%) | YI | Δ (%) |
| 14C | Control | 3.1 | 0.0 | 10.2 | 0.0 | 28.7 | 0.0 | 53.4 | 0.0 | 69.8 | 0.0 |
| 15C | 1% AO-4 | 2.9 | −6.5 | 7.2 | −29.4 | 9.8 | −65.9 | 13.7 | −74.3 | 18.7 | −73.2 |
| 16C | 1% AO-5 | 2.9 | −6.5 | 6.9 | −32.4 | 11.3 | −60.6 | 18.0 | −66.3 | 25.8 | −63.0 |
| 17 | 1% AO-1 | 2.8 | −9.7 | 4.7 | −53.9 | 5.9 | −79.4 | 7.1 | −86.7 | 8.3 | −88.1 |
| 18 | 1% AO-7 | 2.8 | −9.7 | 6.0 | −41.2 | 9.0 | −68.6 | 15.0 | −71.9 | 21.8 | −68.8 |
| 19 | AO-1, AO-4 | 2.9 | −6.5 | 4.8 | −52.9 | 6.1 | −78.7 | 7.6 | −85.8 | 9.5 | −86.4 |
| | 0.5% each | | | | | | | | | | |

The 200° C. oven aging data presented in Table 4 demonstrate that stabilized spandex polymer or prepolymer compositions using AO-1 and AO-7 (Examples 17-18) exhibit consistently lower yellowness index values compared to spandex polymer or prepolymer stabilized with traditional stabilizers AO-4 and AO-5 (Examples 15C and 16C) under the conditions examined. The yellowness index of spandex polymer or prepolymer compositions stabilized with AO-1 and AO-7 are found to range from approximately 10% less than the control in the as-processed condition to approximately 69-88% less than the control after aging for 20 minutes. In contrast, the yellowness index of spandex polymer or prepolymer stabilized with AO-4 and AO-5 is found to range from approximately 7% less than the control in the as-processed condition to approximately 63-73% less than the control after aging for 20 minutes. Additionally, spandex polymer or prepolymer compositions stabilized with a combination of AO-1 and AO-4 (Example 19) exhibit yellowness index values that are comparable to, and in many cases lower than, the individual antioxidants, particularly AO-4 and AO-5. In Example 19, yellowness index is found to range from approximately 7% less than the control in the as-processed condition to approximately 86% less than the control after 20 minutes.

### Examples 20-32

Examples 20-32 illustrate the effect of stabilizer on the color of stabilized spandex polymer or prepolymer compositions during oven aging at 120° C. Color is evaluated at time intervals of approximately 21, 45, 117, and 189 hours. The color of spandex polymer or prepolymer compositions stabilized with AO-1, AO-2, and AO-3 are compared to spandex polymer or prepolymer compositions stabilized with traditional stabilizers AO-4, AO-5, and AO-6. Additionally, the color of spandex polymer or prepolymer compositions stabilized with combinations of AO-1, AO-2, and AO-3 with AO-4, AO-5, and AO-6 are evaluated. The results of these evaluations are presented in Table 5 below.

TABLE 5
OVEN AGING AT 120° C. - YELLOWNESS INDEX VERSUS AGING TIME

| | | Aging Time (hours) | | | | | | | | | |
|---|---|---|---|---|---|---|---|---|---|---|---|
| | Sample | 0 | | 21 | | 45 | | 117 | | 189 | |
| No. | Wt % | YI | Δ (%) | YI | Δ (%) | YI | Δ (%) | YI | Δ (%) | YI | Δ (%) |
| 20C | Control | 3.0 | 0.0 | 7.2 | 0.0 | 20.7 | 0.0 | 45.6 | 0.0 | 59.7 | 0.0 |
| 21C | 1% AO-4 | 2.8 | −6.7 | 5.2 | −27.8 | 7.8 | −62.3 | 16.0 | −64.9 | 23.4 | −60.8 |
| 22C | 1% AO-5 | 3.1 | 3.3 | 5.5 | −23.6 | 7.8 | −62.3 | 15.6 | −65.8 | 22.6 | −62.1 |
| 23C | 1% AO-6 | 2.9 | −3.3 | 5.2 | −27.8 | 7.3 | −64.7 | 15.3 | −66.4 | 22.4 | −62.5 |
| 24 | 1% AO-1 | 2.9 | −3.3 | 4.4 | −38.9 | 6.5 | −68.6 | 11.4 | −75.0 | 17.2 | −71.2 |
| 25 | 1% AO-2 | 3.0 | 0.0 | 4.6 | −36.1 | 6.5 | −68.6 | 11.8 | −74.1 | 18.3 | −69.3 |
| 26 | 1% AO-3 | 2.8 | −6.7 | 4.7 | −34.7 | 6.4 | −69.1 | 11.0 | −75.9 | 16.7 | −72.0 |
| 27 | AO-1, AO-4 | 3.0 | 0.0 | 4.5 | −37.5 | 6.3 | −69.6 | 11.7 | −74.3 | 17.5 | −70.7 |
| | 0.5% each | | | | | | | | | | |
| 28 | AO-1, AO-5 | 2.1 | −30.0 | 4.7 | −34.7 | 6.5 | −68.6 | 11.9 | −73.9 | 18.2 | −69.5 |
| | 0.5% each | | | | | | | | | | |
| 29 | AO-1, AO-6 | 3.1 | 3.3 | 5.0 | −30.6 | 7.0 | −66.2 | 12.8 | −71.9 | 19.0 | −68.2 |
| | 0.5% each | | | | | | | | | | |
| 30 | AO-2, AO-4 | 2.9 | −3.3 | 4.4 | −38.9 | 6.3 | −69.6 | 10.9 | −76.1 | 17.6 | −70.5 |
| | 0.5% each | | | | | | | | | | |
| 31 | AO-3, AO-4 | 2.9 | −3.3 | 4.6 | −36.1 | 6.5 | −68.6 | 10.9 | −76.1 | 17.3 | −71.0 |
| | 0.5% each | | | | | | | | | | |
| 32 | AO-1, AO-4, | 2.9 | −3.3 | 4.4 | −38.9 | 6.3 | −69.6 | 11.6 | −74.6 | 17.8 | −70.2 |
| | (0.375% each) and | | | | | | | | | | |
| | 0.25% HN-150 | | | | | | | | | | |

The 120° C. oven aging data presented in Table 5 demonstrate that spandex polymer or prepolymer stabilized compositions using AO-1, AO-2, and AO-3 (Examples 24-26) exhibit lower yellowness index values compared to spandex polymer or prepolymer stabilized with traditional stabilizers AO-4, AO-5, and AO-6 (Examples 21C-23C) under nearly all conditions examined. The yellowness index of spandex polymer or prepolymer compositions stabilized with AO-1, AO-2, and AO-3 are found to range from approximately 0-7% less than the control in the as-processed condition to approximately 69-71% less than the control after aging for 189 hours. In contrast, the yellowness index of spandex polymer or prepolymer stabilized with AO-4, AO-5, and AO-6 are found to range from approximately 3% greater than the control to 7% less than the control in the as-processed condition to approximately 62% less than the control after aging for 189 hours. Additionally, spandex polymer or prepolymer compositions stabilized with a combination of AO-1 with AO-4, AO-5, and AO-6 (Examples 27-29) and AO-1, AO-2, and AO-3 with AO-4 (Examples 30-32) exhibit yellowness index values that are comparable to, and in some cases lower than, those observed in the individual antioxidants, particularly AO-4 and AO-5. In Examples 27-32, the yellowness index is found to range from approximately 3% greater than the control to 30% less than the control in the as-processed condition to approximately 68-71% less than the control after 189 hours.

### Examples 33-38

Examples 33-38 illustrate the effect of stabilizer on the color of stabilized spandex polymer or prepolymer compositions during oven aging at 120° C. Color is evaluated at time intervals of approximately 24, 48, 71, 92, and 163 hours. The color of spandex polymer or prepolymer compositions stabilized with AO-1 and AO-7 are compared to spandex polymer or prepolymer compositions stabilized with traditional stabilizers AO-4 and AO-5. Additionally, the color of spandex polymer or prepolymer compositions stabilized with a combination of AO-1 and AO-4 is evaluated. The results of these evaluations are presented in Table 6 below.

TABLE 6
OVEN AGING AT 120° C. - YELLOWNESS INDEX VERSUS AGING TIME

| | | Aging Time (hours) | | | | | | | | | | | |
|---|---|---|---|---|---|---|---|---|---|---|---|---|---|
| | | 0 | | 24 | | 48 | | 71 | | 92 | | 163 | |
| No. | Sample | YI | Δ (%) | YI | Δ (%) | YI | Δ (%) | YI | Δ (%) | YI | Δ (%) | YI | Δ (%) |
| 33C | Control | 2.9 | 0.0 | 11.0 | 0.0 | 23.3 | 0.0 | 31.9 | 0.0 | 38.8 | 0.0 | 57.5 | 0.0 |
| 34C | 1% AO-4 | 3.1 | 6.9 | 5.4 | −50.9 | 8.3 | −64.4 | 10.9 | −65.8 | 13.7 | −64.7 | 22.8 | −60.3 |
| 35C | 1% AO-5 | 2.7 | −6.9 | 4.4 | −60.0 | 6.4 | −72.5 | 8.4 | −73.7 | 10.5 | −72.9 | 18.2 | −68.3 |
| 36 | 1% AO-1 | 2.9 | 0.0 | 4.4 | −60.0 | 5.6 | −76.0 | 6.6 | −79.3 | 7.7 | −80.2 | 11.6 | −79.8 |
| 37 | 1% AO-7 | 2.9 | 0.0 | 4.9 | −55.5 | 7.2 | −69.1 | 9.3 | −70.8 | 11.9 | −69.3 | 19.9 | −65.4 |
| 38 | AO-1, AO-4 | 2.9 | 0.0 | 4.5 | −59.1 | 5.8 | −75.1 | 6.8 | −78.7 | 8.0 | −79.4 | 12.4 | −78.4 |
| | 0.5% each | | | | | | | | | | | | |

The 120° C. oven aging data presented in Table 6 illustrates the degree to which stabilized spandex polymer or prepolymer compositions using AO-1 and AO-7 (Examples 36-37) exhibit lower yellowness index values compared to spandex polymer or prepolymer stabilized with traditional stabilizers AO-4 and AO-5 (Examples 34C and 35C) under the conditions examined, particularly as the aging time increases. The yellowness index of spandex polymer or prepolymer compositions stabilized with AO-1 and AO-7 are found to range from approximately unchanged from the control in the as-processed condition to approximately 60-68% less than the control after aging for 163 hours. In contrast, the yellowness index of spandex polymer or prepolymer compositions stabilized with AO-4 and AO-5 are found to range from approximately 7% greater than the control to 7% less than the control in the as-processed condition to approximately 65-73% less than the control after aging for 189 hours. Additionally, spandex polymer or prepolymer compositions stabilized with a combination of AO-1 with AO-4 (Example 38) exhibit yellowness index values that are comparable to the individual antioxidants, and in some cases lower than, those observed in the individual antioxidants, particularly AO-4 and AO-5. In Example 38, yellowness index is found to range from approximately unchanged the control in the as-processed condition to approximately 78% less than the control after 163 hours.

### Examples 39-51

Examples 39-51 illustrate the effect of stabilizer on the color of stabilized spandex polymer or prepolymer compositions during NOₓ oven aging at 60° C. Color is evaluated at time intervals of approximately 7, 25, and 49 hours. The color of spandex polymer or prepolymer compositions stabilized with AO-1, AO-2, and AO-3 are compared to spandex polymer or prepolymer compositions stabilized with traditional stabilizers AO-4, AO-5, and AO-6. Additionally, the color of spandex polymer or prepolymer compositions stabilized with combinations of AO-1, AO-2, and AO-3 with AO-4, AO-5, and AO-6 are evaluated. Further, a stabilized spandex polymer or prepolymer composition with AO-1 and AO-4 further comprising NOₓ stabilizer HN-150 is evaluated. The results of these tests are presented in Table 7 below.

TABLE 7
NO_{X} OVEN AGING AT 60° C. - YELLOWNESS
INDEX VERSUS AGING TIME

| | | Aging Time (hours) | | | | | | | |
|---|---|---|---|---|---|---|---|---|---|
| | Sample | 0 | | 7 | | 25 | | 49 | |
| No. | Wt % | YI | Δ (%) | YI | Δ (%) | YI | Δ (%) | YI | Δ (%) |
| 39C | Control | 3.1 | 0.0 | 7.6 | 0.0 | 27.2 | 0.0 | 47.4 | 0.0 |
| 40C | 1% AO-4 | 2.8 | −9.7 | 4.3 | −43.4 | 11.6 | −57.4 | 24.3 | −48.7 |
| 41C | 1% AO-5 | 3.1 | 0.0 | 5.5 | −27.6 | 16.9 | −37.9 | 29.7 | −37.3 |
| 42C | 1% AO-6 | 3.0 | −3.2 | 5.2 | −31.6 | 15.2 | −44.1 | 28.3 | −40.3 |
| 43 | 1% AO-1 | 3.0 | −3.2 | 4.8 | −36.8 | 12.5 | −54.0 | 25.4 | −46.4 |
| 44 | 1% AO-2 | 3.0 | −3.2 | 4.6 | −39.5 | 13.4 | −50.7 | 26.7 | −43.7 |
| 45 | 1% AO-3 | 2.9 | −6.5 | 4.6 | −39.5 | 14.1 | −48.2 | 27.4 | −42.2 |
| 46 | AO-1, AO-4 | 3.0 | −3.2 | 4.2 | −44.7 | 11.5 | −57.7 | 24.6 | −48.1 |
| | 0.5% each | | | | | | | | |
| 47 | AO-1, AO-5 | 2.7 | −12.9 | 5.2 | −31.6 | 15.5 | −43.0 | 28.8 | −39.2 |
| | 0.5% each | | | | | | | | |
| 48 | AO-1, AO-6 | 3.0 | −3.2 | 4.6 | −39.5 | 13.4 | −50.7 | 26.4 | −44.3 |
| | 0.5% each | | | | | | | | |
| 49 | AO-2, AO-4 | 3.0 | −3.2 | 4.4 | −42.1 | 12.2 | −55.1 | 24.3 | −48.7 |
| | 0.5% each | | | | | | | | |
| 50 | AO-3, AO-4 | 3.0 | −3.2 | 4.0 | −47.4 | 12.0 | −55.9 | 25.8 | −45.6 |
| | 0.5% each | | | | | | | | |
| 51 | AO-1, AO-4, | 2.9 | −6.5 | 3.6 | −52.6 | 7.9 | −71.0 | 13.9 | −70.7 |
| | (0.375% each) and | | | | | | | | |
| | 0.25% HN-150 | | | | | | | | |

The 60° C. NOₓ oven aging data presented in Table 7 demonstrate the degree to which stabilized spandex polymer or prepolymer compositions using AO-1, AO-2, and AO-3 (Examples 43-45) exhibit lower yellowness index values compared to spandex polymer or prepolymer stabilized with traditional stabilizers AO-4, AO-5, and AO-6 (Examples 40C-42C) under the conditions examined. The yellowness index of spandex polymer or prepolymer compositions stabilized with AO-1, AO-2, and AO-3 are found to range from approximately 3-7% less than the control in the as-processed condition to approximately 42-46% less than the control after aging for 49 hours. In contrast, the yellowness index of spandex polymer or prepolymer stabilized with AO-4, AO-5, and AO-6 are found to range from 0-10% less than the control in the as-processed condition to 3749% less than the control after aging for 49 hours. Additionally, spandex polymer or prepolymer compositions stabilized with a combination of AO-1 with AO-4, AO-5, and AO-6 (Examples 46-48) and AO-2, and AO-3 with AO-4 (Examples 49-50) exhibit yellowness index values that are comparable to, and in some cases lower than, those observed in the individual antioxidants. In Examples 46-50, the yellowness index is found to range from approximately 3-13% less than the control in the as-processed condition to approximately 39-49% less than the control after 49 hours.

Further, the stabilized spandex polymer or prepolymer composition with AO-1 and AO-4 further comprising NOₓ stabilizer HN-150 (Example 51) exhibited yellowness index values that are comparable to, and in most cases significantly lower than, those observed in the individual antioxidants. In Example 51, the yellowness index is found to range from approximately 7% less than the control in the as-processed condition to approximately 71% less than the control after 49 hours.

### Examples 52-57

Examples 52-57 illustrate the effect of stabilizer on the color of stabilized spandex polymer or prepolymer compositions during NOₓ oven aging at 60° C. Color is evaluated at time intervals of approximately 6, 12, 18, and 24 hours. The color of spandex polymer or prepolymer compositions stabilized with AO-1, and AO-7 are compared to spandex polymer or prepolymer compositions stabilized with traditional stabilizers AO-4 and AO-5. Additionally, the color of spandex polymer or prepolymer compositions stabilized with combinations of AO-1 and AO-4 are evaluated. The results of these evaluations are presented in Table 8 below.

TABLE 8
NO_{X} OVEN AGING AT 60° C. - YELLOWNESS INDEX VERSUS AGING TIME

| | | Aging Time (min) | | | | | | | | | |
|---|---|---|---|---|---|---|---|---|---|---|---|
| | | 0 | | 6 | | 12 | | 18 | | 24 | |
| No. | Sample | YI | Δ (%) | YI | Δ (%) | YI | Δ (%) | YI | Δ (%) | YI | Δ (%) |
| 52C | Control | 3.4 | 0.0 | 14.7 | 0.0 | 25.3 | 0.0 | 34.5 | 0.0 | 36.9 | 0.0 |
| 53C | 1% AO-4 | 2.9 | −14.7 | 6.2 | −57.8 | 11.7 | −53.8 | 18.8 | −45.5 | 23.1 | −37.4 |
| 54C | 1% AO-5 | 2.8 | −17.6 | 10.0 | −32.0 | 19.3 | −23.7 | 28.1 | −18.6 | 30.8 | −16.5 |
| 55 | 1% AO-1 | 2.8 | −17.6 | 6.0 | −59.2 | 11.3 | −55.3 | 18.9 | −45.2 | 23.4 | −36.6 |
| 56 | 1% AO-7 | 3.0 | −11.8 | 10.0 | −32.0 | 17.3 | −31.6 | 25.4 | −26.4 | 28.4 | −23.0 |
| 57 | AO-1, AO-4 | 2.8 | −17.6 | 6.4 | −56.5 | 12.8 | −49.4 | 20.1 | −41.7 | 24.8 | −32.8 |
| | 0.5% each | | | | | | | | | | |

The 60° C. NOₓ oven aging data presented in Table 8 demonstrate the extent to which stabilized spandex polymer or prepolymer compositions using AO-1 and AO-7 (Examples 55-56) exhibit lower yellowness index values compared to spandex polymer or prepolymer stabilized with traditional stabilizers AO-4 and AO-5 (Examples 53C-54C) under the conditions examined. The yellowness index of spandex polymer or prepolymer compositions stabilized with AO-1 and AO-7 is found to range from approximately 12-18% less than the control in the as-processed condition to approximately 23-37% less than the control after aging for 24 hours. In contrast, the yellowness index of spandex polymer or prepolymer stabilized with AO-4 and AO-5 is found to range from 15-18% less than the control in the as-process condition to approximately 17-37% after aging for 24 hours. Additionally, spandex polymer or prepolymer compositions stabilized with a combination of AO-1 with AO-4 (Examples 57) exhibit yellowness index values that are comparable to those observed in the individual antioxidants. In Example 57, the yellowness index is found to range from approximately 18% less than the control in the as-processed condition to approximately 33% less than the control after 49 hours.

### Example 58

Example 5 is repeated except that AO-1 is replaced with AO-8. Similar results are obtained.

### Example 59

The compound of formula (AO-10) is prepared as follows: To a stirring mixture of about 13.2 g of [4-(1,1-dimethylethyl)-3-hydroxy-2,6-dimethylphenyl]methyl 2-propenoate, (Evans, et al. GB 2,103,624) and about 100 mL MIBK is added about 5 g of triethylamine. The contents are cooled to about 0-5° C. and then about 10 g of n-dodecanethiol is dropwise added. The reaction mixture is allowed to warm to room temperature. About 2 mL of additional TEA is added and the mixture is gradually heated to reflux. The reaction completion is followed by HPLC. The reaction mixture is then concentrated to remove volatiles. The residue is purified by column chromatography over silica gel using a mixture of methylene chloride and hexane in about a 1:1 ratio. The purified material thus obtained is characterized by NMR and mass to be the desired product.

### Example 60

The compound of formula (AO-11) is prepared as follows: The method described in Example 59 above to prepare the compound of formula (AO-10) is used to make the compound of formula (AO-11) with a difference that about 14.3 g of n-octadecanethiol is used instead of about 10 g of n-dodecanethiol. The crude product is similarly purified by column chromatography over silica gel using a mixture of methylene chloride and hexane in about a 1:1 ratio. The purified product thus obtained is characterized by NMR and mass to be the desired product.

### Example 61

The compound of formula (AO-12) is prepared as follows: To a solution of about 2.0 g (6.7 mmol) of 2-(1,1-dimethylethyl)-4-mercapto-6-methylphenol, [Fujisawa, et al. Synthesis (1973), (1), 38-9] in about 20 mL of THF is added about 1.31 g (6.7 mmol) of [4-(1,1-dimethylethyl)-3-hydroxy-2,6-dimethylphenyl]methyl 2-propenoate, (Evans, et al. GB 2,103,624) and about 20 mg of triethylamine. The resulting mixture is heated at about 50° C. for about 16 hours, and the solvent removed by rotary evaporation. The residue is flash chromatographed using 70% methylene chloride/hexane, giving about 1.5 g of product as a white solid, m.p. 129-134° C. The purified material thus obtained is characterized by NMR and mass to be the desired product.

### Example 62

The compound of formula (AO-13) is prepared as follows: To a solution of about 1.07 g (5.5 mmol) of 2-(1,1-dimethylethyl)-4-mercapto-6-methylphenol, [Fujisawa, et al. Synthesis (1973), (1), 38-9] in about 5 mL THF is added about 0.27 g (1.05 mmol) of 1,2-ethanediylbis(oxy-2,1-ethanediyl) 2-propenoate (available from Sartomer Company, Inc., Exton, Pa.) and about 20 mg of triethylamine. The mixture is heated at about 50° C. for about 24 hours, and the solvent removed by rotary evaporation. The residue is flash chromatographed using 20% ethyl acetate/methylene chloride, giving about 0.55 g of a colorless oil. The purified material thus obtained is characterized by NMR and mass to be the desired product.

### Example 63

The composition of formula (AO-14) is prepared as follows: 500 g of AO-1 is added to a 4 L plastic container, followed by 500 g of AO-4. The container is shaken by hand, then mixed on a roll blender for about 8 hours. HPLC and NMR indicated a homogeneous mixture. The melting point of the physical blend was found to be about 101.9° C. to 165.8° C. The solid state 13 C NMR showed one CO2R resonance at about 179 ppm. The IR showed one ester C═O stretch at approx. 1705 cm⁻¹.

### Examples 64-67

The composition of formula (AO-15) is prepared as follows:

### Example 64

Procedure 1: A mixture of about 100 g AO-4 and about 100 g of AO-1 is dissolved in about 400 mL acetone. To it is then added about 600 mL methylcyclohexane. The solution is concentrated to remove acetone and some methylcyclohexane. Total volume distilled is about 500 mL. The remaining solution is allowed to cool to ambient temperature. The recrystallized material is filtered and washed with 2× ca. 150 mL methylcyclohexane. The filtered product is then dried in a vacuum oven at about 40° C. for approx. 3 hr and then at about 60-70° C. for approx. 6 hr.

### Example 65

Procedure 2: The above procedure in Example 64 is repeated but with a difference that methylcyclohexane is replaced by cyclohexane. Thus a mixture of about 100 g AO-4 and about 100 g of AO-1 is dissolved in about 400 mL acetone. To it is then added about 600 mL cyclohexane. The solution is concentrated to remove acetone and some methylcyclohexane. Total volume distilled is about 500 mL. The remaining solution is allowed to cool to ambient temperature. The recrystallized material is filtered and washed with 2× ca. 150 mL cyclohexane. The filtered product is then dried in a vacuum oven at about 40° C. for approx. 3 hr and then at about 60-70° C. for approx. 6 hr.

### Example 66

Procedure 3: The above procedure in Example 64 is repeated but with a difference that the mixture of AO-4 and AO-1 is dissolved in methylene chloride instead of acetone. Thus a mixture of about 100 g AO-4 and about 100 g of AO-1 is dissolved in about 400 mL methylene chloride. To it is then added about 600 mL methylcyclohexane. The solution is concentrated to remove methylene chloride. The remaining solution is allowed to cool to ambient temperature. The recrystallized material is filtered and washed with 2× ca. 150 mL methylcyclohexane. The filtered product is then dried in a vacuum oven at about 40° C. for approx. 3 hr and then at about 60-70° C. for ca. 6 hr.

### Example 67

Procedure 4: The above procedure in Example 66 is repeated but with a difference that methylcyclohexane is replaced by cyclohexane. Thus a mixture of about 100 g AO-4 and about 100 g of AO-1 is dissolved in about 400 mL methylene chloride. To it is then added about 600 mL cyclohexane. The solution is concentrated to remove methylene chloride. The remaining solution is allowed to cool to ambient temperature. The recrystallized material is filtered and washed with 2× ca. 150 mL cyclohexane. The filtered product is then dried in a vacuum oven at about 40° C. for approx. 3 hr and then at about 60-70° C. for approx. 6 hr. The melting point of the co-crystal was found to be about 164.4° C. to 166.0° C. The solid state ¹³C NMR showed two CO₂R resonances, at approx. 176 and 179 ppm. The IR showed two ester C═O stretches, at approx. 1705 cm⁻¹ and at 1733 cm⁻¹.

TABLE 9
OVEN AGING AT 200° C. - YELLOWNESS INDEX VERSUS AGING TIME

| | | Aging Time (minutes) | | | | | | | | | |
|---|---|---|---|---|---|---|---|---|---|---|---|
| | Sample | 0 | | 5 | | 10 | | 15 | | 20 | |
| No. | Wt % | YI | Δ (%) | YI | Δ (%) | YI | Δ (%) | YI | Δ (%) | YI | Δ (%) |
| | Control | 3.4 | 0.0 | 11.2 | 0.0 | 33.8 | 0.0 | 59.2 | 0.0 | 76.1 | 0.0 |
| | 1% AO-4 | 3.3 | −2.9 | 8.2 | −26.8 | 11.1 | −67.2 | 15.2 | −74.3 | 21.5 | −71.7 |
| | 1% AO-10 | 3.2 | −5.9 | 4.6 | −58.9 | 5.3 | −84.3 | 5.9 | −90.0 | 6.4 | −91.6 |
| | 1% AO-11 | 3.2 | −5.9 | 4.8 | −57.1 | 5.4 | −84.0 | 5.9 | −90.0 | 6.5 | −91.5 |
| | 1% AO-1 | 3.1 | −8.8 | 4.8 | −57.1 | 5.8 | −82.8 | 7.0 | −88.2 | 8.4 | −67.7 |
| | 1% AO-12 | 2.8 | −17.6 | 5.2 | −53.6 | 6.4 | −81.1 | 7.5 | −87.3 | 9.1 | −88.0 |
| | 1% AO-13 | 3.0 | −11.8 | 5.2 | −53.6 | 6.3 | −81.4 | 7.4 | −87.5 | 9.1 | −88.0 |

TABLE 10
OVEN AGING AT 120° C. - YELLOWNESS INDEX VERSUS AGING TIME

| | | Aging Time (hours) | | | | | | | | | |
|---|---|---|---|---|---|---|---|---|---|---|---|
| | Sample | 0 | | 30 | | 80 | | 98 | | 164 | |
| No. | Wt % | YI | Δ (%) | YI | Δ (%) | YI | Δ (%) | YI | Δ (%) | YI | Δ (%) |
| | Control | 3.2 | 0.0 | 15.7 | 0.0 | 40.3 | 0.0 | 59.6 | 0.0 | 66.3 | 0.0 |
| | 1% AO-4 | 3.5 | −9.4 | 7.1 | −54.8 | 13.5 | −66.5 | 23.2 | 61.1 | 27.4 | −58.7 |
| | 1% AO-10 | 3.1 | −3.1 | 4.7 | −70.1 | 6.7 | −83.4 | 10.3 | −82.7 | 12.3 | −81.4 |
| | 1% AO-11 | 2.9 | −9.4 | 4.7 | −70.1 | 6.7 | −83.4 | 10.1 | −83.0 | 12 | −81.9 |
| | 1% AO-1 | 2.9 | −9.4 | 5 | −68.2 | 7.8 | −80.6 | 12.2 | −79.5 | 14.8 | −77.6 |
| | 1% AO-12 | 3.1 | −3.1 | 5.5 | −65.0 | 8.7 | −78.4 | 10.3 | −82.7 | 15.0 | −77.4 |
| | 1% AO-13 | 3.3 | 3.1 | 5.6 | −64.3 | 8.9 | −77.9 | 101 | −83.0 | 14.9 | −77.5 |

TABLE 11
COLOR STABILITY AND PHYSICAL CONSTANT COMPARISON BETWEEN PHYSICAL BLEND
(AO-14) AND COCRYSTAL (AO-15) AND INDIVIDUAL COMPONENTS (AO-1 AND AO-4)

| | | Melting | Infrared | Solid State | 40° C. Oven Aging Time (days) | | | | |
|---|---|---|---|---|---|---|---|---|---|
| | | Point | Carbonyl | ¹³C NMR | 0 | 55 | | 95 | |
| No. | Sample | (° C.) | Str. (cm-1) | Carbonyl (ppm) | Hazen value | Hazen Value | Δ (%) | Hazen value | Δ (%) |
| | AO-14 | ca. 101.9-165 | ca. 1705 | ca. 179 | 33 | 59 | 78.8 | 179 | 442.4 |
| | AO-15 | ca. 164.4-166 | ca. 1705, 1733 | ca. 176, 179 | 27 | 41 | 51.8 | 49 | 81.5 |

Although the foregoing description has shown, described, and pointed out the fundamental novel features of the present teachings, it will be understood that various omissions, substitutions, and changes in the form of the detail of the apparatus as illustrated, as well as the uses thereof, may be made by those skilled in the art, without departing from the scope of the present teachings. Consequently, the scope of the present teachings should not be limited to the foregoing discussion, but should be defined by the appended claims.

1. A stabilized spandex polymer or prepolymer composition comprising spandex polymer or prepolymer and a stabilizing amount of a compound of formula (I):
LEₙ (I)
wherein:
E is an end group, L is a linking group, and n is an integer in the range of 1 to 10 that represents the number of F end groups attached to the linking group L;
each of the E end groups are independently represented by
-{A(CH₂)ₘ₂—S}ₐ(CH₂)ₘ₁—[CZQ]_{q}-Ar
where q=0-10,
a is zero or one, with a first proviso that for a=zero, n is 2 and the linking group L is 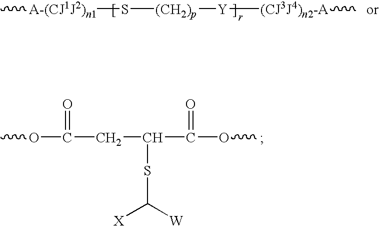
and with a second proviso that for a one, n=2 to 10 and the linking group L is an n-valent optionally substituted C₁-C₃₀ hydrocarbyl group;
and with a third proviso that when n=1, L is a terminal group
L=H—(CJ¹J²)ₙ₁-S—[(CH₂)ₚY]ᵣ—(CJ³J⁴)ₙ₂-A-
A represents an ester linkage;
p is an integer in the range of 2 to 4;
r is zero or an integer in the range of 1 to 3;
Y is O or S;
X and W are each independently H, Ar¹ (CH₂)ₙ₃—, or Ar²(CH₂)ₙ₄—O₂C—(CH₂)ₙ₅—;
J¹, J², J³, and J⁴ are each independently H, C₁₋₁₀ alkyl or C₆₋₁₀ aryl;
n1, n2, and m2 are each independently an integer in the range of 1 to 20, with a fourth proviso that n1, n2, and m2 are selected so that the compound of formula (I) does not include a covalent bond between sulfur and oxygen;
n3, n4, n5, and m1 are each independently zero or an integer in the range of 1 to 20;
each Z is independently an optionally substituted aryl or H;
each Q is independently an optionally substituted C₁₋₆ alkyl or H;
Ar, Ar¹, and Ar² are each independently selected from 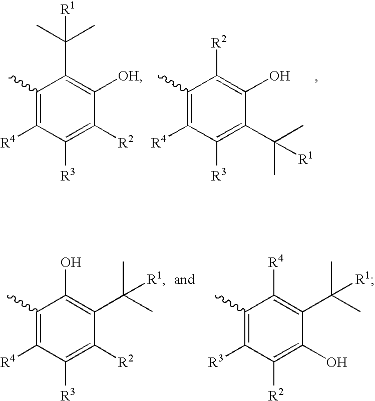
wherein R¹ is selected from C₁-C₆ alkyl, C₆-C₁₀ aryl and C₇-C₁₂ arylalkyl; and
wherein R², R³ and R⁴ are each independently H or C₁-C₆ alkyl;
with a fifth proviso that formula (I) does not include the following compounds:

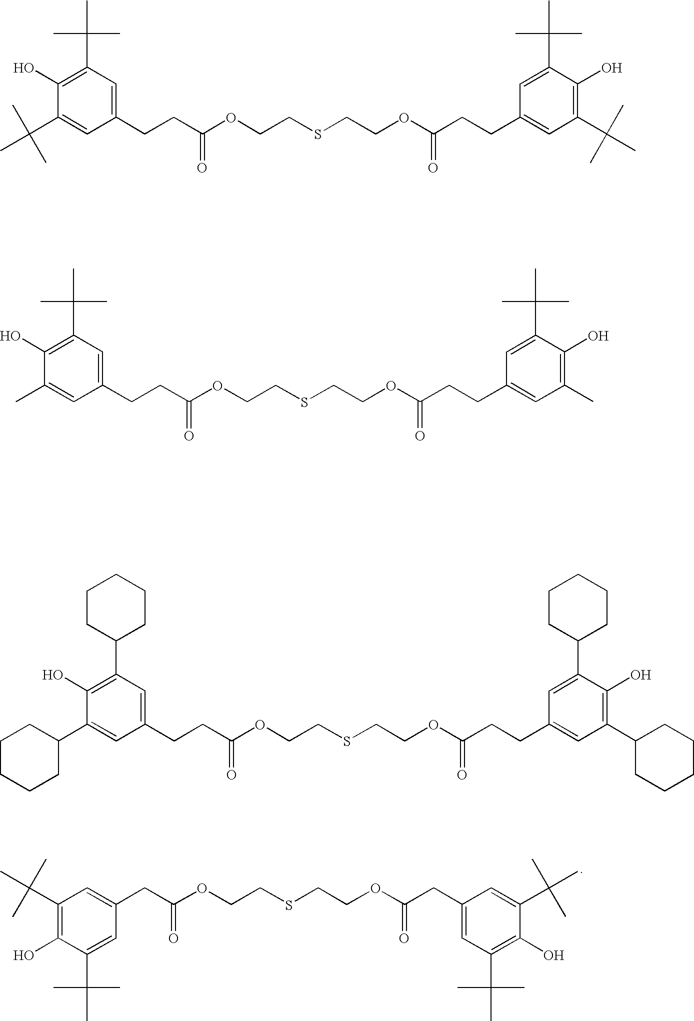

2. The composition of claim 1, wherein Z is aryl, a is zero and m1 is zero.

3. The composition of claim 1, wherein a is zero and wherein r and A are selected so that the linking group L is

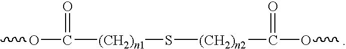

4. The composition of claim 3, wherein the compound of the formula (I) is

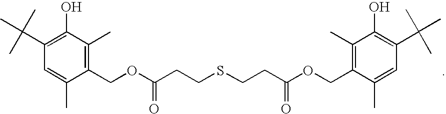

5. The composition of claim 1, wherein a=zero, n is 2, the linking group L is 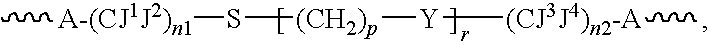
r=0, n1=n2=2, and at least one of J¹, J², J³, and J⁴ is methyl.

6. The composition of claim 5, wherein the compound of the formula (I)

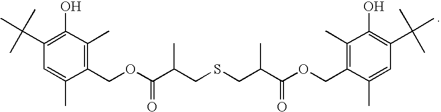

7. The composition of claim 1, wherein a is zero and wherein r and A are selected so that the linking group L is

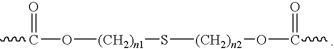

8. The composition of claim 1, wherein a is zero, r is an integer in the range of 1 to 3, and A is selected so that the linking group L is

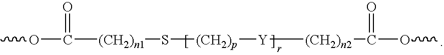

9. The composition of claim 8, wherein the compound of the formula (I) is

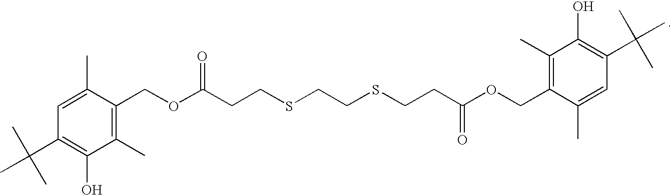

10. The composition of claim 1, wherein a is zero, r is an integer in the range of 1 to 3, and A is selected so that the linking group L is

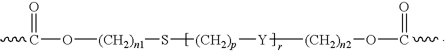

11. The composition of claim 1, wherein a is zero and the linking group L is

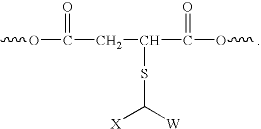

12. The composition of claim 11, wherein Z and Q are both H.

13. The composition of claim 1, wherein a is one and at least one A is selected so that at least one end group E is

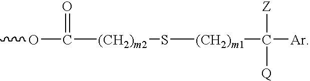

14. The composition of claim 13, wherein the compound of the formula (I) is

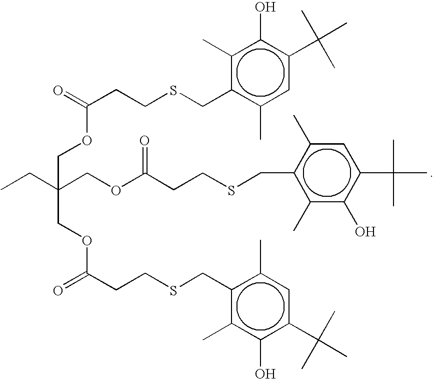

15. The composition of claim 13, wherein the compound of the formula (I) is

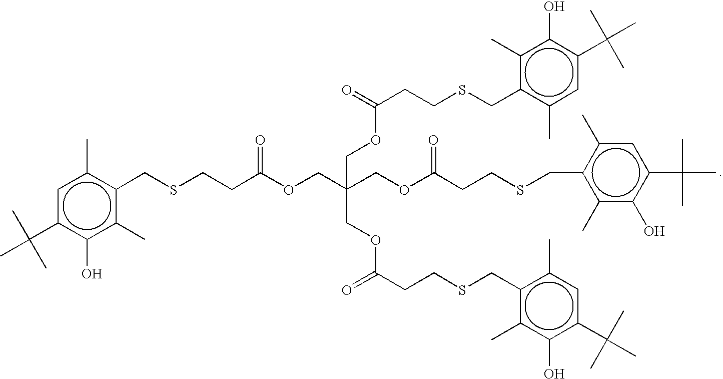

16. The composition of claim 1, wherein a is one and at least one A is selected so that at least one end group E is

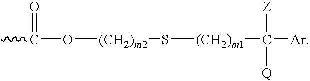

17. The composition of claim 1 wherein the compound of formula (I) is

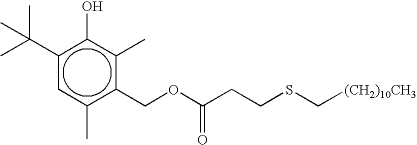

18. The composition of claim 1 wherein the compound of formula (I) is

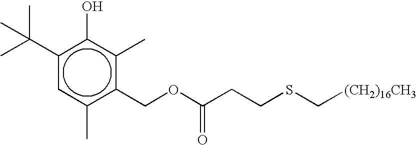

19. The composition of claim 1 wherein —H—(CJ¹J²)ₙ₁ in the third proviso is substituted by Ar¹ and the compound of formula (I) is

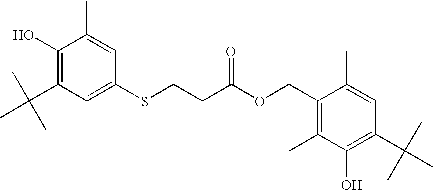

20. The composition of claim 1 wherein the compound of formula (I) is

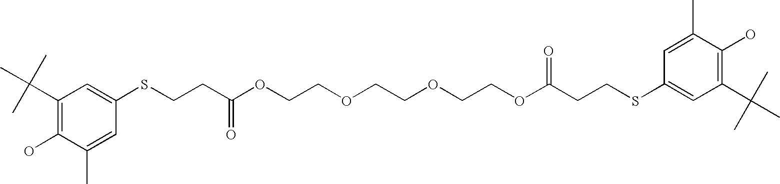

21. A composition according to claim 1, wherein the stabilizing amount of the compound of formula (I) is in the range of about 0.01% to about 10%, by weight based on total weight of the composition.

22. The composition of claim 21, wherein the stabilizing amount of the compound of formula (I) is in the range of about 0.05% to about 5%, by weight based on total weight of the composition.

23. A composition according to claim 1, further comprising a substantially sulfur-free hindered phenol antioxidant.

24. The composition of claim 23, wherein the substantially sulfur-free hindered phenol antioxidant is selected from 1,3,5-tris(4-tert-butyl-3-hydroxy-2,6 dimethylbenzyl)-1,3,5-triazine-2,4,6-(1H,3H,5H)-trione, tiethyleneglycol bis-3-(3-tert-butyl-4-hydroxy-5-methylphenyl)propionate, 9-bis(2-(3-(3-tert-butyl-4-hydroxy-5-methylphenyl)propionyloxy)-1,1-dimethylethyl)-2,4,8,10-tetraoxaspiro[5.5]undecane 6,2,6-di-tert-butyl-4-methylphenol, 2-tert-butyl-4,6-di-methylphenol, 2,6-di-tert-butyl-4-ethylphenol, 2,6-di-tert-butyl-4-n-butylphenol, 2,6-di-tert-butyl-4-isobutylphenol, 2,6-dicyclopentyl-4-methylphenol, 2-(α-methylcyclohexyl)-4,6-dimethylphenol, 2,6-dioctadecyl-4-methylphenol, 2,4,6-tricyclohexylphenol, 2,6-di-tert-butyl-4-meth-oxymethylphenol, ortho-nonylphenol, 2,6-di-nonyl-4-methylphenol, 2,4-dimethyl-6-(1′-methylundec-1′-yl)phenol, 2,4-dimethyl-6-(1′-methylheptadec-1′-yl)phenol, 2,4-dimethyl-6-(1′-methyltridec-1′-yl)phenol, ethylene-1,2-bis(3,3-bis[3-t-butyl-4 hydroxyphenyl]butyrate), 1,1-bis(2-methyl-5-t-butyl-4-hydroxyphenyl)butane, and 1,1,3-tris(2-methyl-5-t-butyl-4-hydroxyphenyl)butane.

25. The composition of claim 24, wherein the substantially sulfur-free hindered phenol antioxidant is selected from 1,3,5-tris(4-tert-butyl-3-hydroxy-2,6 dimethylbenzyl)-1,3,5-triazine-2,4,6-(1H,3H,5H)-trione, triethyleneglycol bis-3-(3-tert-butyl-4-hydroxy-5-methylphenyl)propionate, and 9-bis(2-(3-(3-tert-butyl-4-hydroxy-5-methylphenyl)propionyloxy)-1,1-dimethylethyl)-2,4,8,10-tetraoxaspiro[5.5]undecane 6.

26. A composition according to claim 1, further comprising an NOx stabilizer selected from N,N′-(methylenedi-1,4-phenylene)bis[2,2-dimethyl hydrazine carboxamide], a poly(dialkyl aminoethylmethacrylate), and a lauric acid hydrazide reaction product with bisphenol A diglycidyl ether.

27. The composition of claim 26, wherein the NOx stabilizer is N,N′-(methylenedi-1,4-phenylene)bis[2,2-dimethyl hydrazine carboxamide].

28. A composition of according to claim 1, wherein R² is H or C₁-C₆ n-alkyl.

29. A method for making a composition one according to claim 1, comprising intermixing the spandex polymer or prepolymer and the compound of the formula (I).

30. A compound chosen from any one of the following formulas

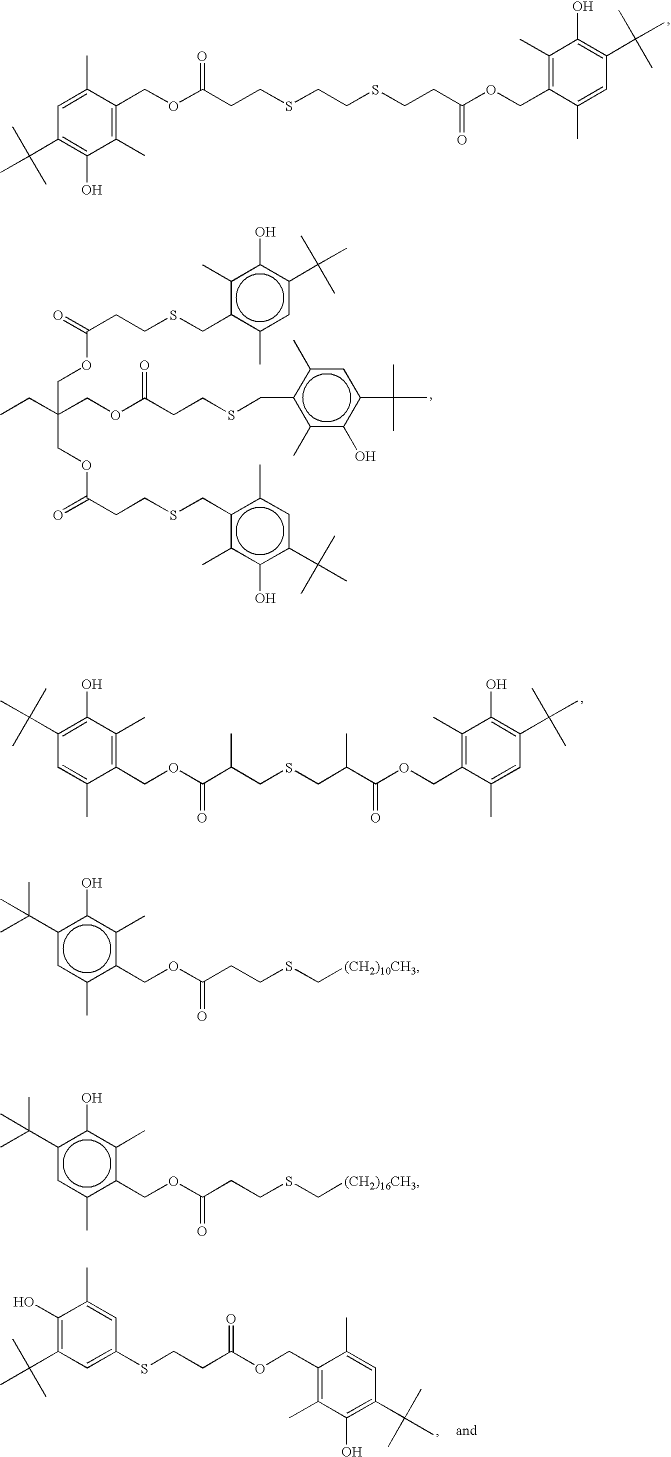

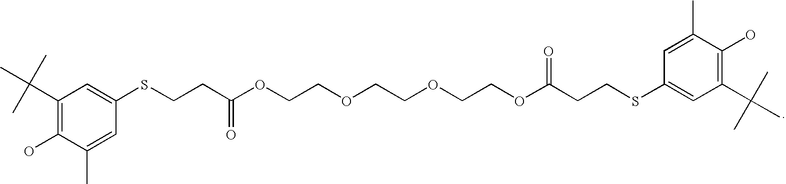

31. A composition comprising a polymer and a compound of claim 30.

32-43. (canceled)

44. A antioxidant complex (AO-15) comprising 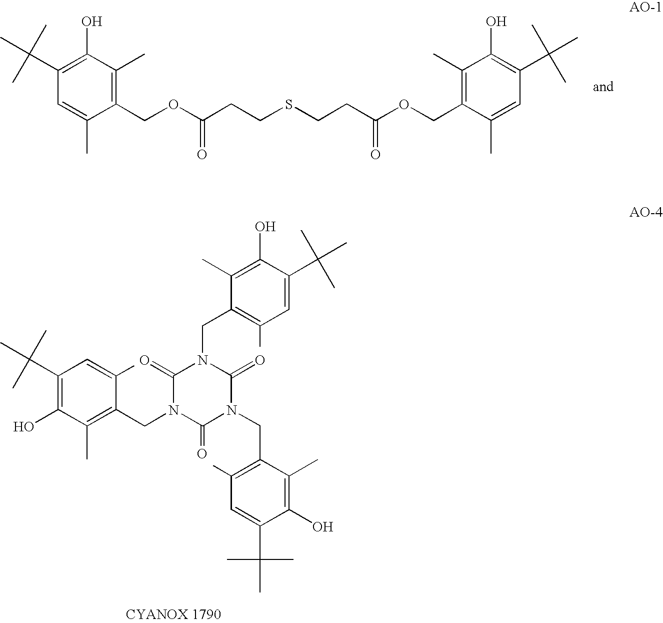
wherein said complex is prepared by co-crystallization from a solution containing AO-1 and AO-4.

45. The complex according to claim 44, wherein the ratio of AO-1 to AO-4 is from about 4:1 to about 1:4.

46. The complex according to claim 45, wherein the ratio of AO-1 to AO-4 is 1:1.
